(19) United States
(12) Patent Application Publication
(10) Pub. No.: US 2025/0107892 A1
(43) Pub. Date: Apr. 3, 2025

(54) PROSTHETIC HEART VALVE THAT REDUCES NATIVE ANNULUS

(71) Applicant: EDWARDS LIFESCIENCES CORPORATION, Irvine, CA (US)

(72) Inventors: David L. Hauser, Newport Beach, CA (US); David Robert Landon, Huntington Beach, CA (US); Rani Abdullah Mahmoudi, Huntington Beach, CA (US); Eric Robert Dixon, Villa Park, CA (US)

(21) Appl. No.: 18/981,479

(22) Filed: Dec. 14, 2024

Related U.S. Application Data
(63) Continuation of application No. PCT/US2023/025489, filed on Jun. 16, 2023.
(60) Provisional application No. 63/352,777, filed on Jun. 16, 2022.

(51) Int. Cl. A61F 2/24

(52) U.S. Cl. CPC A61F 2/2445; A61F 2/2418; A61F 2210/0014; A61F 2220/0016; A61F 2230/005; A61F 2230/0091; A61F 2250/0031

(57) ABSTRACT

A prosthetic heart valve configured to be expanded within a native valve annulus, engage surrounding tissue, and constricted tissue inward to enhance contact between the tissue and the valve. The heart valve may have a structural valve stent with an outer constricting structure having tissue-engaging members or anchors. The constricting structure may be intrinsic or extrinsic. One intrinsic version includes a plurality of arms that are arranged to constrict inward toward a central valve portion, or a fabric structure with tissue anchors and surrounding an inner valve member may be cinched to pull the tissue inward. The anchors may engage tissue around the annulus in the atrium, or surround leaflets in the ventricle, or grasp leaflets from within.

### CROSS-REFERENCE TO PRIORITY APPLICATIONS

This application is a continuation of International Patent Application No. PCT/US2023/025489, filed Jun. 16, 2023, which claims the benefit of U.S. Provisional Application No. 63/352,777 filed Jun. 16, 2022, the entire content of which is hereby incorporated herein by reference.

### TECHNICAL FIELD

The present application relates generally to expandable prosthetic heart valves, such as replacement mitral or tricuspid heart valves, that better secure to and remodel the valve annulus.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered through a catheter or other such access tube.

Expandable valves sometimes do not seal as well against the native annulus as would a surgical valve that is sutured into place, leading to paravalvular leakage. In addition, expandable valves typically push radially outwardly against the surrounding annulus, which can exacerbate the underlying disease and remodel the heart in an undesirable manner. Still further, replacement valves can sometimes contact surrounding tissue (e.g., ventricle walls) with adverse effects. Consequently, there is a need for expandable heart valves that are capable of pulling surrounding tissue inwardly to form a better seal, remodel the heart in a more favorable manner and avoid contact with heart walls.

### SUMMARY

Disclosed herein are expandable prosthetic heart valves, such as replacement mitral or tricuspid heart valves, that better secure to and in some cases remodel the valve annulus. The expandable prosthetic heart valves may have structure that anchors to the surrounding annulus tissue and pulls the tissue inwardly, often remodeling the annulus and potentially the ventricle below the annulus. Advantages to these features include minimizing the device profile needed to treat a very large annulus, providing an adaptable platform to treat a range of annuli with one or very few valve sizes, and, depending on the anchoring mechanism, the procedure can largely be guided via fluoroscopy.

One example of a prosthetic heart valve assembly is for replacing a native mitral or tricuspid valve. The prosthetic heart valve assembly includes a self-expandable stent having a main body with an inlet end portion and an outlet end portion. The stent may be made from a shape memory material such as Nitinol. A valve portion is positioned within a passageway of the main body for permitting the flow of blood through the passageway in only one direction, thereby replacing the function of the native valve. At least one anchor and preferably multiple anchors are disposed along an exterior surface of the prosthetic heart valve assembly for engaging surrounding tissue. The prosthetic heart valve assembly is adapted to reduce a diameter of the native valve annulus by pulling surrounding tissue inwardly. Reducing the diameter of the annulus may provide numerous advantages, such as treating the underlying disease (e.g., performing annuloplasty), reducing the device profile in the body, and creating a better seal around the exterior of the device.

The prosthetic heart valve assembly may include an annular flange extending radially outwardly from the main body, such as from the inlet end portion. One or more tissue-engaging anchors may be disposed on the annular flange, such as along a lower surface or on the perimeter. The anchors may comprise barbs, helical screws, or any other suitable tissue-engaging mechanism. The annular flange is coupled to the main body and configured to transition from a large diameter to a smaller diameter for constricting the annulus of the native valve. In one embodiment, the annular flange may comprise a plurality of radially extending arms, wherein each arm is configured to reduce in length for pulling tissue inwardly and thereby constricting the annulus of the native valve. The reduction in length of an arm may be achieved using a bioresorbable material that initially maintains the arm in an elongated state and resorbs in the body for allowing the arm to transition to a shortened state.

In a prosthetic heart valve assembly having an annular flange, a cinching mechanism may connect each of the anchors. The cinching mechanism may be adapted to pull the anchors radially inwardly for constricting the annulus of the native valve.

In a prosthetic heart valve assembly having an annular flange, the anchors may be deployed separately from the heart valve such that the anchors are not integrated into the heart valve.

In a prosthetic heart valve assembly having an annular flange, the annular flange may be coupled to the main body and configured to reduce in diameter during radial expansion of the main body.

In a prosthetic heart valve assembly having an annular flange, the annular flange may comprise a plurality of spirally arranged arms that when expanded have tips that together define a circle of revolution having a diameter D₁, and wherein rotation of the valve portion about its axis pulls the arms inward such that the tips together define a constricted diameter D₂.

The prosthetic heart valve assembly may further comprise one or more ventricular anchors extending from the main body, such as from the outlet end portion. The ventricular anchors may be shaped for capturing a native valve leaflet between the ventricular anchor and the main body of the stent. Alternatively, the ventricular anchors may be shaped for placement inside the native leaflets and may have an outer surface for engaging surrounding tissue. In preferred embodiments, the ventricular anchors transition (i.e., flip) from a generally straight shape to a curved shape upon deployment.

Another example of a prosthetic heart valve assembly comprises a stent having a main body, a valve portion positioned within a passageway of the main body, and a plurality of anchors disposed along an exterior surface of the stent for engaging surrounding tissue. The stent may be made from a shape memory material such as Nitinol. The stent may be adapted to be radially over-expanded by an expansion mechanism to a diameter larger than its shape set diameter for ensuring that the anchors firmly engage and/or penetrate surrounding tissue. The expansion mechanism is then removed for allowing the stent to return toward its shape set diameter. As the stent returns to its shape set diameter (i.e., reduces in diameter), it pulls the surrounding tissue inwardly.

The expansion mechanism may be a balloon or any other suitable mechanism for temporarily expanding a stent. The stent may comprise an annular flange extending radially outwardly from the main body, wherein anchors are disposed along a surface of the annular flange. Alternatively, the stent may comprise at least one ventricular anchor extending from the main body. The ventricular anchor is desirably shaped for capturing a native valve leaflet between the ventricular anchor and the main body of the stent. Still further, the stent may comprise an annular flange extending radially outwardly from the inlet end portion of the main body and at least one ventricular anchor for capturing a native valve leaflet between the ventricular anchor and the main body of the stent. A fabric seal may cover at least a portion of the stent.

Another prosthetic heart valve assembly may be configured for replacing a native mitral or tricuspid valve. The prosthetic valve assembly includes a self-expandable valve stent made from a shape memory material and covered with fabric, the valve stent having a tubular valve portion with an inlet end portion and an outlet end portion and a peripheral flange when expanded formed by an array of struts or arms extending radially outward from and connected to the inlet end portion, the array having an intrinsic radial constriction mechanism and tissue-engaging members. A valve portion is positioned within a passageway of the main body, wherein the valve portion comprises a plurality of leaflets made from pericardium, wherein the valve portion permits flow of blood through the passageway in one direction for replacing the function of the native valve. The prosthetic heart valve assembly is adapted to anchor the tissue-engaging members to tissue surrounding the valve annulus to radially constrict or shrink the annulus of the native valve upon deployment.

The array may comprise petal-shaped struts coupled to the valve portion such that expansion of the valve portion causes the struts to radially constrict. Alternatively, the array may comprise struts that are configured to curl outward and then inward toward the valve portion when an external restraint around the valve is removed from the inflow end, the struts having barbs on outer tips that define the tissue-engaging members.

The array may comprise a plurality of radially extending arms each of which has a constriction mechanism for reducing a length of the arm built in. The constriction mechanism may include an extended structure biased to a constricted length and held extended by a bioresorbable suture. The constriction mechanism may alternatively include an extended structure biased to a constricted length and held extended by a stiffening wire. Only some of the radially extending arms may have a tissue-engaging member thereon, and the radially extending arms may have dissimilar lengths.

In yet another embodiment, the array may comprise a plurality of spirally arranged arms that when expanded have tips that together define a circle of revolution having a diameter D₁, and wherein rotation of the valve portion about its axis pulls the arms inward such that the tips together define a constricted diameter D₂.

In another variation, the valve stent may have an outer anchor stent and an inner valve stent, and the array comprises a plurality of radial arms extending from struts connected just at a lower end of the anchor stent, wherein rotation of the anchor stent subsequent to anchoring of barbs at tips of the radial arms causes the struts to rotate into helical shapes and pull the tips inwardly, the inner valve stent then being expanded within the outer anchor stent.

Another example of a prosthetic heart valve assembly for replacing a native mitral or tricuspid valve includes a self-expandable valve stent made from a shape memory material and covered with fabric, wherein the valve stent has a tubular valve portion with an inlet end portion and an outlet end portion. An array of arms extends radially outward from and connect to the outlet end portion of the valve portion and may be configured to bend (e.g., 180°) toward the inlet end portion. The array has an intrinsic radial constriction mechanism and tissue-engaging members. A valve portion is positioned within a passageway of the main body, wherein the valve portion comprises a plurality of leaflets made from pericardium, wherein the valve portion permits flow of blood through the passageway in one direction for replacing the function of the native valve. The prosthetic heart valve assembly is adapted to anchor the tissue-engaging members to native valve leaflets to pull the leaflets towards the tubular valve portion upon deployment.

In another example, the intrinsic radial constriction mechanism may comprise a stiffening tube mounted around a lower U-bend on each of the arms, the stiffening tubes having a greater radius of curvature than the U-bends to force the arms to an outward position, and the stiffening tubes being bioresorbable after a certain time within the body to permit the arms to revert to a radially inward shape.

In another example, the intrinsic radial constriction mechanism may comprise one or more stiffening plugs mounted positioned within recesses on an inner radius of each of the arms, the stiffening plugs holding the arms in an outward position, and the stiffening plugs being bioresorbable after a certain time within the body to permit the arms to revert to a radially inward shape.

A system for constricting an implantable prosthetic heart valve configured for implant at a native mitral or tricuspid valve annulus includes a heart valve having a self-expandable valve stent covered with fabric, the valve stent having a tubular valve portion and a valve member with leaflets mounted therein, the valve further having a peripheral flange when expanded comprising a generally annular fabric-covered array of struts or arms extending radially outward from and connected to an inflow end of the valve portion, the array having tissue-engaging members configured to grasp and anchor to tissue surrounding the valve annulus. An extrinsic radial constriction mechanism is provided for pulling tissue surrounding the valve annulus inwardly subsequent to deploying the tissue-engaging members. The extrinsic radial constriction mechanism may comprise a flexible cinch having a length sufficient to extend from outside the body and threaded to connect and slide through each of the tissue-engaging members, wherein tension on the cinch constricts the array and pulls tissue surrounding the valve annulus inwardly.

The tissue-engaging members may comprise valve anchors separate from the peripheral flange and deployed through the peripheral flange once the heart valve is seated at the valve annulus.

The array may comprise a plurality of radially extending arms and the tissue-engaging members comprise anchoring barbs secured to outer ends of at least some of the arms.

Another system for constricting an implantable prosthetic heart valve configured for implant at a native mitral or tricuspid valve annulus includes a heart valve having a self-expandable valve stent covered with fabric, the valve stent having a tubular valve portion and a valve member with leaflets mounted therein, the valve further having a peripheral flange when expanded comprising a generally annular fabric-covered array of struts or arms extending radially outward from and connected to an inflow end of the valve portion, the array having tissue-engaging members configured to grasp and anchor to tissue surrounding the valve annulus, and wherein the peripheral flange is discontinuous at a free edge exposed on an inflow side thereof such that it has a variable circumferential size. An extrinsic radial constriction mechanism is provided for pulling tissue surrounding the valve annulus inwardly subsequent to deploying the tissue-engaging members, the extrinsic radial constriction mechanism comprising a pair of flexible tethers having a length sufficient to extend from outside the body and threaded to connect and slide through a pair of radially-spaced anchors embedded through the peripheral flange into annulus tissue and extending circumferentially along the peripheral flange to attach to the free edge thereof, wherein tension on the tethers circumferentially constricts the peripheral flange and pulls tissue surrounding the valve annulus inward.

Another system for constricting an implantable prosthetic heart valve configured for implant at a native mitral or tricuspid valve annulus includes a heart valve having a self-expandable valve stent covered with fabric, the valve stent having a tubular valve portion and a valve member with leaflets mounted therein, the valve further having a peripheral flange when expanded comprising a generally annular fabric-covered array of struts or arms extending radially outward from and connected to an inflow end of the valve portion. At least one tissue anchor is separate from the prosthetic heart valve is configured to be embedded within tissue surrounding the valve annulus. An extrinsic radial constriction mechanism is configured to pull tissue surrounding the valve annulus inwardly subsequent to deploying the tissue anchors, the extrinsic radial constriction mechanism comprising a plurality of flexible tethers having a length sufficient to extend from outside the body, extend through the peripheral flange, and extend radially outward to fasten to the tissue anchors, wherein tension on the tethers constricts pulls the tissue anchors and tissue surrounding the peripheral flange inward.

A constricting prosthetic heart valve for implant at a native mitral or tricuspid valve annulus includes a self-expandable valve stent covered with fabric, the valve stent having a tubular valve portion and a valve member with leaflets mounted therein, the valve further having a fabric-covered array of arms extending radially outward from and connected to an outflow end of the valve portion and bent 180° toward an inflow end, the array having tissue-engaging members configured to grasp and anchor to leaflets attached to the valve annulus. An extrinsic radial constriction mechanism is configured to pull leaflets attached to the valve annulus inward subsequent to deploying the tissue-engaging members, the extrinsic radial constriction mechanism comprising a flexible cinch having a length sufficient to extend from outside the body and threaded to connect and slide through and entirely around the fabric covering the array, wherein tension on the cinch constricts the fabric-covered array and pulls the leaflets inward.

Another constricting prosthetic heart valve for implant at a native mitral or tricuspid valve annulus comprises a self-expandable valve stent covered with fabric, the valve stent having a tubular valve portion and a valve member with leaflets mounted therein, the valve further having a fabric-covered outer tube spaced radially outward from the valve portion and connected thereto with upper and lower flexible skirts, the outer tube having tissue-engaging members configured to grasp and anchor to leaflets attached to the valve annulus. An extrinsic radial constriction mechanism is configured to pull leaflets attached to the valve annulus inward subsequent to deploying the tissue-engaging members, the extrinsic radial constriction mechanism comprising a flexible cinch having a length sufficient to extend from outside the body and threaded to connect and slide through and entirely around the fabric covering the outer tube, wherein tension on the cinch constricts the fabric-covered outer tube and pulls the leaflets inward.

A method of constricting a prosthetic heart valve implanted at a native mitral or tricuspid valve annulus includes the step of providing a heart valve having a self-expandable valve stent covered with fabric, the valve stent having a tubular valve portion and a valve member with leaflets mounted therein, the valve portion having a relaxed expanded size, the valve further having a peripheral flange comprising a generally annular fabric-covered array of struts or arms extending radially outward from and connected to an inflow end of the valve portion, the array having tissue-engaging members configured to grasp and anchor to tissue surrounding the valve annulus. The heart valve is crimped around a balloon and constrained in a constricted state within an access sheath. The heart valve is advanced in the constricted state within the access sheath toward the valve annulus. The heart valve is expelled from the access sheath, preferably within the valve annulus. The balloon is inflated beyond the expanded size of the valve portion of the valve stent, thereby anchoring or embedding the tissue-engaging members into tissue surrounding the valve annulus. The balloon is then deflated to permit the valve portion to constrict and pull the peripheral flange inwardly.

Another method of constricting a prosthetic heart valve implanted at a native mitral or tricuspid valve annulus includes providing a self-expandable valve stent covered with fabric, the valve stent having a tubular valve portion and a valve member with leaflets mounted therein, the valve portion having a relaxed expanded size, the valve further having a fabric-covered array of arms extending radially outward from and connected to an outflow end of the valve portion and bent toward an inflow end, the array having tissue-engaging members configured to grasp and anchor to leaflets attached to the valve annulus. The heart valve is prepared for implant by crimping the heart valve around a balloon and constraining the heart valve in a constricted state within an access sheath. The heart valve is advanced in the constricted state within the access sheath to the valve annulus. The heart valve is released from the access sheath within the valve annulus and the balloon is inflated for over-expanding the valve portion of the valve stent (i.e., beyond its shape set diameter), thereby anchoring the tissue-engaging members into the leaflets. The balloon is then deflated to permit the valve portion to constrict to its expanded size and pull the array inward.

A further understanding of the nature and advantages of the invention will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1A is a cutaway view of the human heart in a systolic phase, FIG. 1B is the same view showing a heart with the left ventricle enlarged (dilated), often termed dilated cardiomyopathy (DCM), and FIG. 1C is the same view showing a heart with the right ventricle enlarged, often leading to functional tricuspid regurgitation;

FIG. 2A is a partial sectional view through the left side of a heart showing a mitral valve and annulus above the left ventricular structure, and FIG. 2B is a plan view of the mitral valve showing well-known nomenclature of the annulus and leaflets;

FIG. 3A is a partial sectional view similar to that in FIG. 2A after implantation of an expandable prosthetic heart valve of the present application prior to a final step in deployment, and FIG. 3B is the same view after a final step of radially constricting an outer skirt anchored to the mitral annulus;

FIGS. 4A-4C are perspective and orthogonal views of an exemplary valve stent of the prosthetic heart valve shown in FIGS. 3A and 3B without an outer fabric cover and valve leaflets;

FIG. 5 is a laid-flat view of the valve stent of FIGS. 4A-4C;

FIGS. 6A-6C are sequential views showing just the valve stent being expelled from an access tube to illustrate steps in the deployment thereof;

FIG. 7 is a perspective view of an alternative expandable prosthetic heart valve of the present application having an upper peripheral flange and showing a plurality of anchors prior to deployment through the flange;

FIG. 8A is a sectional view through a left ventricle showing the expandable prosthetic heart valve of FIG. 7 prior to a final step in deployment within the mitral annulus, and FIG. 8B is the same view after a final step of radially constricting the peripheral flange anchored to the mitral annulus;

FIG. 9A is a perspective view of a still further expandable prosthetic heart valve having an upper peripheral flange and showing a plurality of anchors deployed therein, and FIG. 9B shows a final step of circumferentially cinching the peripheral flange to cause radial constriction thereof;

FIG. 10A is an elevational view of an alternative heart valve stent of the present application having a tubular valve portion and an upper peripheral flange with anchoring barbs, while FIG. 10B shows one example of an anchoring barb;

FIG. 10C is an elevational view of a valve stent similar to that in FIG. 10A with a main body and an upper peripheral flange, and also ventricular anchors extending from a lower end of the main body, while FIG. 10D shows the valve stent implanted at a mitral annulus;

FIGS. 11A and 11B are two views illustrating a step in deployment of the valve stent of FIG. 10, wherein expansion of a central portion of the valve stent causes radial constriction of the upper peripheral flange;

FIGS. 12A-12C are sequential views showing a still further valve stent being expelled from an access tube to illustrate steps in the deployment thereof;

FIGS. 13A and 13B are top plan and side elevational views of an exemplary expanded prosthetic heart valve of the present application;

FIG. 13C is an enlarged portion of FIG. 13A illustrating one particular configuration of a constricting arm thereof, and FIG. 13D is an enlarged portion thereof showing a constricting mechanism;

FIG. 14 is a top plan view of an upper peripheral flange of an exemplary stent of the present application, and FIGS. 14A and 14B show actuation of one radial arm thereof before and after radial constriction;

FIG. 14C shows a collapsible radial arm having a stiffening member formed as a bioresorbable rod or stick;

FIG. 15A is a top plan view of an exemplary prosthetic heart valve similar in construction to that of FIG. 13A with a stent frame peripheral flange like that in FIG. 14, and FIG. 15B is a view of the heart valve after radial constriction of the peripheral flange;

FIGS. 16A and 16B are top plan views of another exemplary valve stent having a radially constricting peripheral flange with a series of outer petals;

FIGS. 17A and 17B are top plan views of another exemplary valve stent having a radially constricting peripheral flange with a series of spiral arms;

FIGS. 18A-18C are schematic elevational views of a two-part expandable prosthetic heart valve stent assembly of the present application showing several steps in deployment thereof;

FIG. 19 is elevational view of another valve stent of the present application having radially retractable arms around an upper periphery, and FIG. 19A illustrates retraction of one of the arms utilizing a ratcheting mechanism;

FIGS. 20A and 20B are perspective views of an outer fabric structure useful in constriction of expandable prosthetic heart valves of the present application in both initially deployed and constricted configurations, respectively;

FIG. 21 is a perspective view of an assembly of the outer fabric structure of FIG. 20A with a prosthetic heart valve mounted therein;

FIGS. 22A and 22B are top plan views of the assembly of FIG. 21 before and after radial constriction of the outer fabric structure;

FIG. 23A is elevational view of another valve stent of the present application having radially retractable arms, FIG. 23B is an enlargement of a retraction mechanism, and FIGS. 23C and 23D are schematic views showing operation thereof;

FIG. 24 is elevational view of yet another valve stent of the present application having outer arms that are biased radially inward;

FIGS. 25A and 25B are views of one of the arms of the valve stent in FIG. 24 before and after dissolution of a temporary measure to hold the arms outward;

FIGS. 26A and 26B are views of one of the arms of the valve stent in FIG. 24 before and after dissolution of another temporary measure to hold the arms outward;

FIG. 27 is elevational view of still another valve stent of the present application having outer arms with active tissue anchors, and FIG. 27A is an enlargement of one of the active tissue anchors;

FIGS. 28A-28C are top plan views of one of the active tissue anchors showing several phases of deployment;

FIG. 29 is a partial sectional view through the left side of a heart showing a mitral valve and annulus with pre-installed tissue anchors therein, and an expanded prosthetic heart valve being advanced down an array of sutures to the annulus;

FIG. 30 shows the expanded prosthetic valve advanced nearly down to the annulus causing the pre-installed tissue anchors to be pulled inward;

FIGS. 31A and 31B show engagement between a flat clip on the peripheral flange of the heart valve and one of the tissue anchors;

FIG. 32A shows a valve stent held in an expanded state by a delivery balloon adjacent a target annulus, and FIG. 32B shows the valve stent in an implant position within the annulus with the balloon removed to permit constriction of the annulus;

FIG. 33A shows another type of valve stent held in an expanded state by a delivery balloon adjacent a target annulus, and FIG. 33B shows the valve stent in an implant position within the annulus with the balloon removed to permit constriction of the annulus;

FIG. 34 is a perspective view of a still further heart valve stent having a tubular valve portion and an upper peripheral flange formed by radially-extending arms tipped with anchoring barbs, and FIG. 35 is an isolation of one segment of the valve stent with an anchoring arm;

FIGS. 36A-36C show a sequence of delivering and implanting a heart valve having the stent of FIG. 34 using a tubular sheath;

FIGS. 37A and 37B illustrate one configuration for the anchoring barbs of the stent of FIG. 34 activated by sutures in tension;

FIGS. 38 and 39 illustrate two alternative configurations for the anchoring barbs of the stent of FIG. 34;

FIG. 40 is a perspective view of a heart valve stent similar to that in FIG. 34 but having anchoring barbs on only some of the radially extending arms, and FIG. 40A is an enlargement of an alternative type of anchoring barb; and

FIG. 41 is a perspective view of still another heart valve stent similar to that in FIG. 34 but having radially extending arms of different lengths, and FIG. 41A is an enlargement of another type of anchoring barb.

### DETAILED DESCRIPTION OF CERTAIN EMBODIMENTS

Valve replacement in the mitral or tricuspid annulus is a primary focus of the present application, but certain characteristics of the delivery systems described herein may equally be used for other valve implant locations, and thus the claims should not be constrained to mitral or tricuspid valve replacement unless expressly limited. Replacement heart valves can be delivered to a patient's heart mitral valve annulus or other heart valve location in various manners, such as by open surgery, minimally invasive surgery, and percutaneous or transcatheter delivery through the patient's vasculature. Example transfemoral approaches may be found in U.S. Pat. Nos. 10,004,599 and 10,813,757, the entireties of which are hereby incorporated by reference. All techniques of valve delivery are contemplated by the present application.

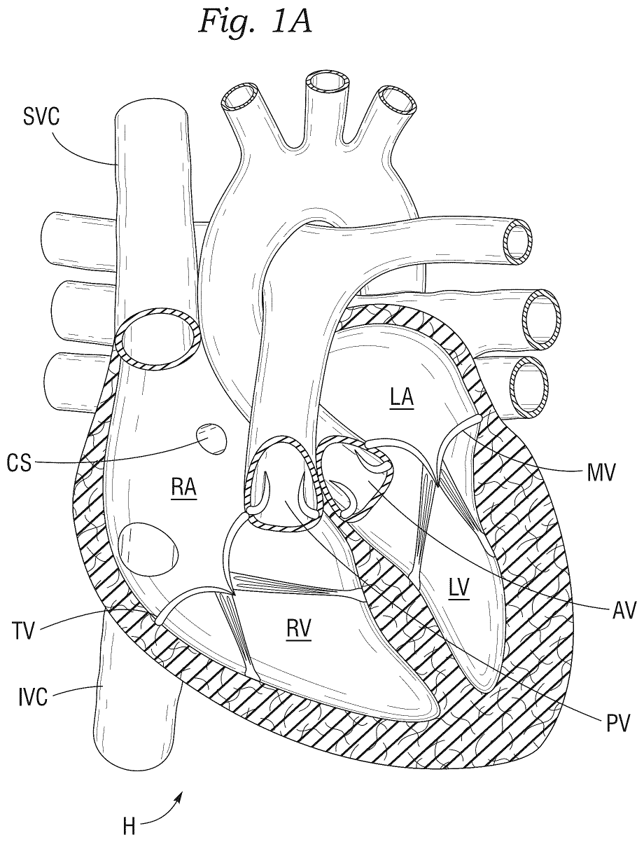

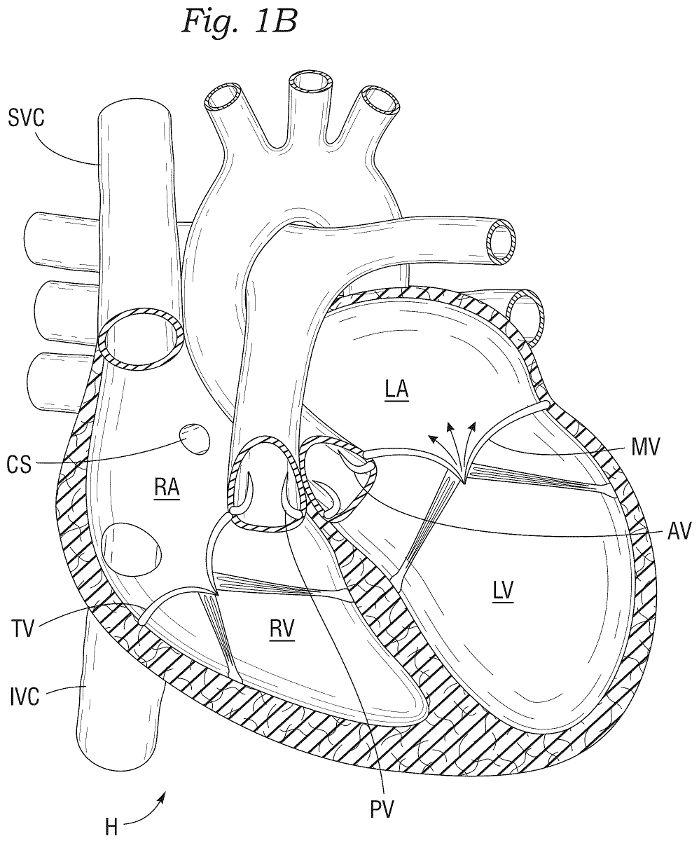

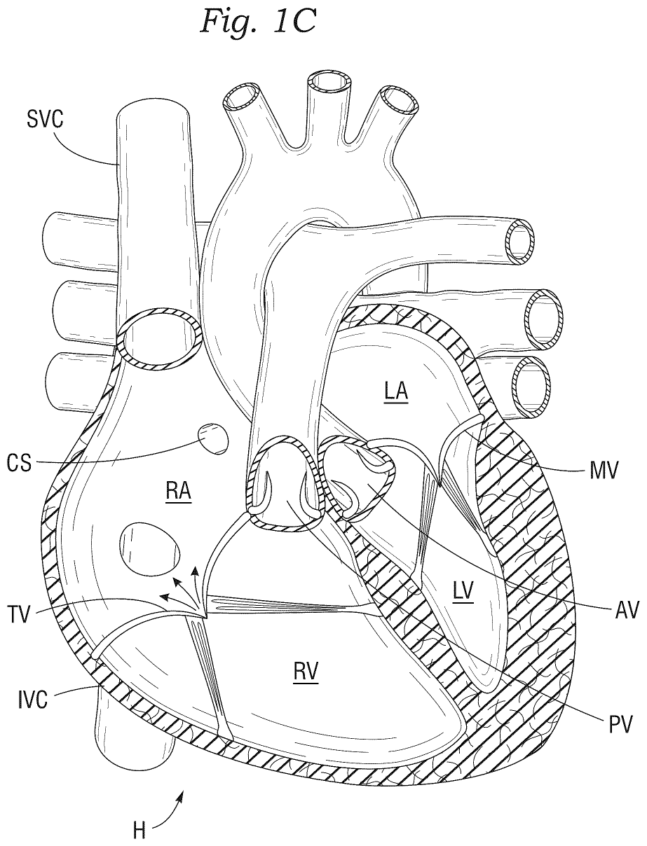

FIG. 1A is a cutaway view of the human heart in a systolic phase. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta (not identified) and the pulmonary valve PV separates the right ventricle from the pulmonary artery (also not identified). Each of these valves has flexible leaflets extending inward across the respective orifices that come together or “coapt” in the flowstream to form the one-way fluid occluding surfaces. The right atrium RA receives deoxygenated blood from the venous system through the superior vena cava SVC and the inferior vena cava IVC, the former entering the right atrium above, and the latter from below. The coronary sinus CS is a collection of veins joined together to form a large vessel that collects deoxygenated blood from the heart muscle (myocardium), and delivers it to the right atrium RA.

During the diastolic phase, or diastole, the venous blood that collects in the right atrium RA is pulled through the tricuspid valve TV by expansion of the right ventricle RV, and likewise oxygenated blood is pulled through the mitral valve MV by expansion of the left ventricle LV. During the higher pressure systolic phase, or systole, seen in FIG. 1A, the heart muscle squeezes both the right ventricle RV and left ventricle LV which forces venous blood through the pulmonary valve PV and pulmonary artery into the lungs and forces arterial blood through the aortic valve AV, ascending aorta and throughout the body. It is during this high pressure phase that the leaflets of the tricuspid valve TV and mitral valve MV close to prevent the blood from regurgitating back into the respective right and left atriums RA, LA.

FIG. 1B is the same view as FIG. 1A but shows a heart with the left ventricle LV enlarged (dilated), often termed dilated cardiomyopathy (DCM). Such a condition tends to pull apart the mitral valve MV leaflets so they do not close properly, often leading to mitral regurgitation as shown by the jets of blood escaping through the ostensibly closed MV. Regurgitation reduces the pressure generated in the left ventricle LV, and thus the pressure gradient across the aortic valve AV, which diminishes the amount of blood pumped through the body.

In a similar manner, FIG. 1C is the same view showing a heart with the right ventricle RV enlarged; a condition caused by various factors. The tricuspid valve TV annulus dilates outwards towards the anterior and posterior RV free wall, which can lead to functional tricuspid regurgitation, as shown. Reduced pumping output through the pulmonary valve PV to the lungs reduces the overall efficiency of the oxygenation process, leading to various cascading health problems.

The present application provides a number of solutions to the issues raised by either dilated cardiomyopathy (DCM) or functional tricuspid regurgitation. In general, implanting replacement expandable prosthetic heart valves within an enlarged annulus may lead to secondary issues of paravalvular leakage due to the widened annulus. First of all, commercial expandable prosthetic heart valves have cylindrical exteriors which do not closely match the non-circular peripheral shapes of either the mitral or tricuspid annulus. Secondly, the stent frame of expandable prosthetic heart valves is either self-expanding or balloon-expanding. While balloon-expanding stent frames exert more outward force on the annulus, neither type of stent frame is immune from potential leakage around their exteriors.

The present application contemplates expandable prosthetic heart valves that grab onto surrounding annulus tissue and pull it radially inward against the outside of the valve. This not only helps reduce paravalvular leakage, but over time may also contribute to remodeling of the annulus and the subvalvular structures. For example, an expandable heart valve of the type described herein implanted in the tricuspid annulus pulls the annulus radially inward, which also pulls the adjacent wall of the right ventricle RV inward. It is believed that over time the improved blood flow and radial constriction of the tricuspid annulus helps remodel the right ventricle RV and reduce dilation thereof. Another potential benefit of the expandable heart valves described herein is the ability to minimize the device profile required to treat a very large annulus. That is, instead of selecting an oversized heart valve, a properly sized valve is used which then pulls the annulus in around it. Furthermore, the constricting heart valves described herein provide an adaptable valve platform in which a small number of sizes of valves may be used to treat a wide range of annulus sizes. Current prosthetic heart valves are available from 19 mm to a maximum size of 31 or 33 mm, in 2 mm increments. Constricting heart valves disclosed herein may be provided in size 19 mm for annuluses between 19-21 mm, size 23 mm for annuluses between 23-25 mm, and so on, thus reducing the inventory of valve sizes needed. Finally, the various heart valves described herein can be implanted with the aid of fluoroscopy, as with current expandable heart valves.

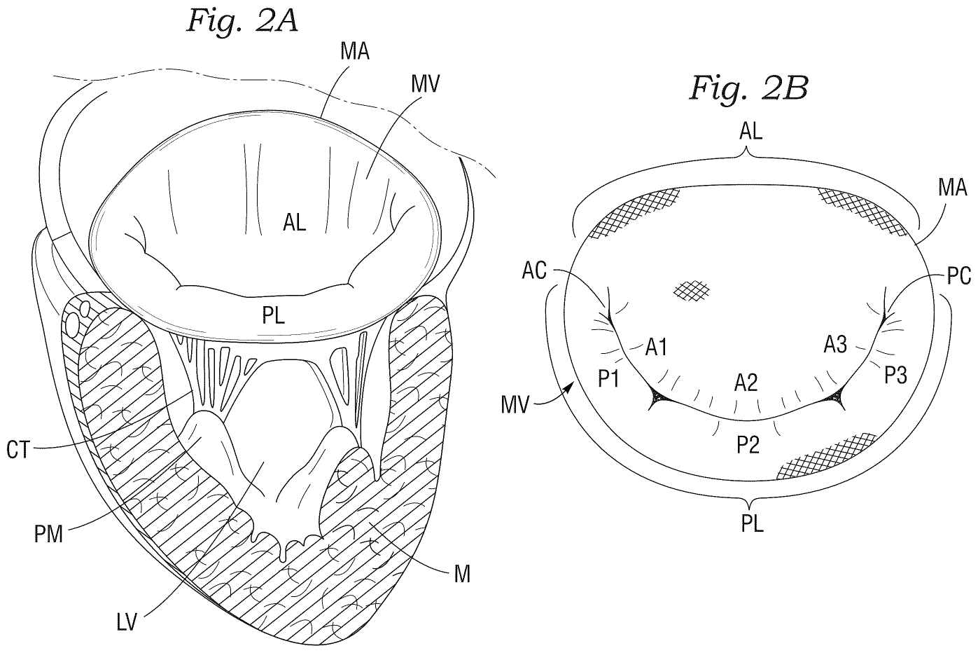

FIG. 2A is a partial sectional view through the left side of a heart showing a mitral valve and annulus above the left ventricular structure, and FIG. 2B is a plan view of the mitral valve showing well-known nomenclature of the annulus and leaflets. The mitral valve MV primarily comprises a pair of coapting leaflets—an anterior leaflet AL and a posterior leaflet PL—secured around their outer edges to a fibrous mitral annulus MA. The inner edges of the anterior and posterior leaflets AL, PL connect to string-like chordae tendineae CT that extend down into the left ventricle LV and are tethered at papillary muscles PM that extend upward from the muscular myocardium M defining the left ventricular cavity. As the main outlet pumping chamber of the heart, the myocardium M contracts in systole which reduces tension in the chordae tendineae CT and permits the leaflets AL, PL to come together or coapt.

As seen in FIG. 2B, the surrounding mitral annulus MA is often described as D-shaped with a somewhat straighter side adjacent the anterior leaflet AL and a more rounded or convex side adjacent the posterior leaflet PL. The leaflets are shaped such that the line of coaptation resembles a smile that approximately parallels the posterior aspect of the mitral annulus MA. The anterior leaflet AL spans a smaller peripheral aspect around the mitral annulus MA than the posterior leaflet PL, but the anterior leaflet AL has a single cusp with a convex free edge that extends farther into the orifice defined by the mitral annulus MA. Typically, the anterior leaflet AL has three labeled regions or cusps A1, A2, A3 around its periphery. The posterior leaflet PL, on the other hand, is typically divided by creases into three cusps P1, P2, P3 around its periphery and has a generally concave free edge. Two commissures—an anterior commissure AC and a posterior commissure PC-generally define the intersection of the line of coaptation between the two leaflets AL, PL and the mitral annulus MA. The tricuspid valve TV is not detailed but is often described as an irregular ovoid shape. The tricuspid annulus has three dissimilar leaflets extending inward for co-optation.

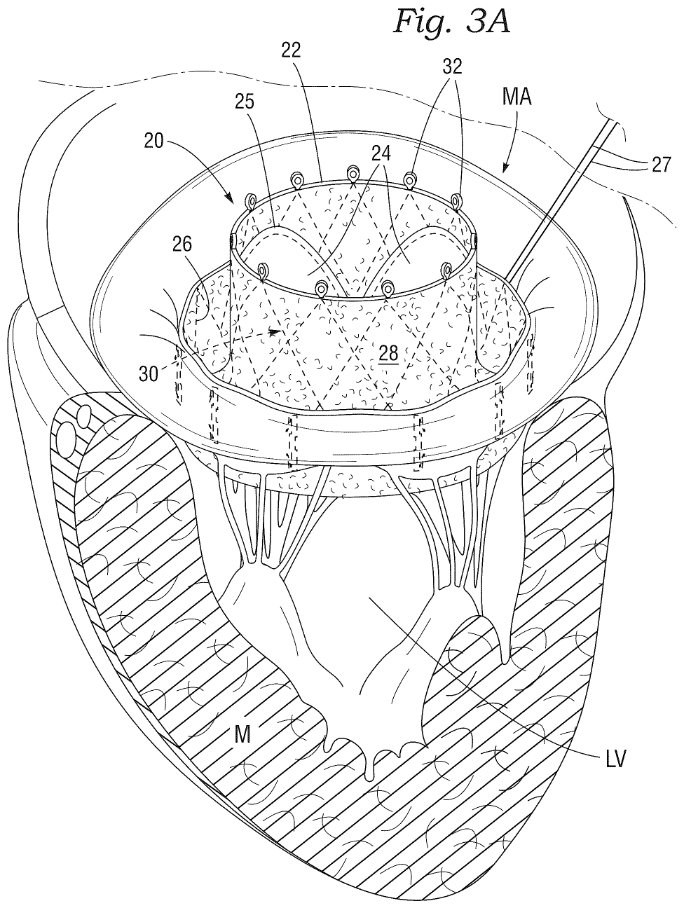

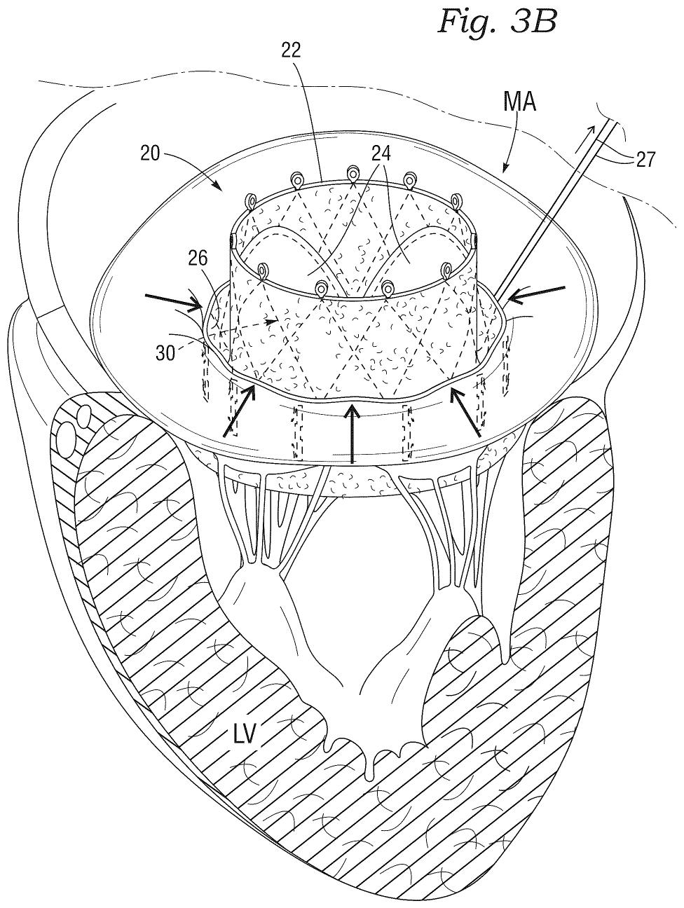

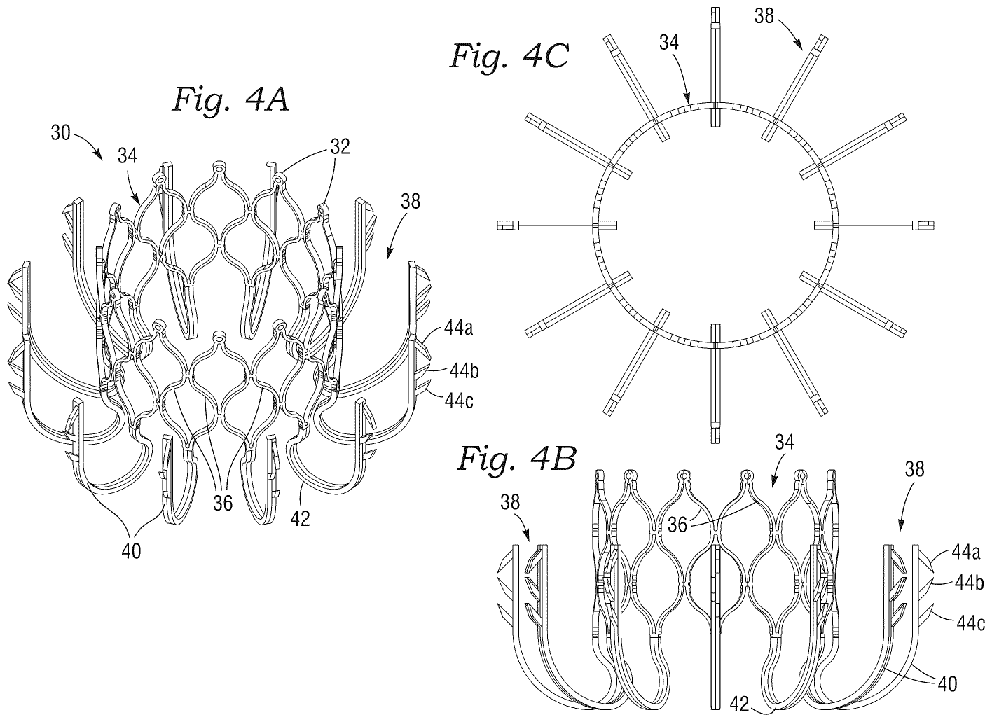

It should be understood that any of the heart valve stents disclosed herein may be shaped to conform to the D-shaped mitral annulus MA as seen in FIG. 2B. That is, all of the stents are shown with tubular main bodies that contain and support the leaflets. Instead of a circular plan view, such as seen in FIGS. 3A-3B and 4C, the main body may be D-shaped, with a somewhat straighter side to be implanted adjacent the anterior leaflet AL and a more rounded or convex side to be implanted adjacent the posterior leaflet PL.

Several of the constricting heart valve embodiments described herein are shown being implanted within the mitral valve MV, though it should be understood that the same valves can also be utilized at the tricuspid valve TV. Indeed, all of the heart valve embodiments described herein can be interchangeably used at either of the atrioventricular valves.

FIG. 3A is a partial sectional view of the mitral annulus MA and left ventricle LV similar to that in FIG. 2A after implantation of an expandable prosthetic heart valve 20 of the present application, but prior to a final step in deployment. FIG. 3B is the same view after a final step of radially constricting an outer skirt anchored to the mitral annulus. Reference is also made to FIGS. 4A-4C which show an exemplary valve stent without the fabric covering and valve leaflets.

The prosthetic heart valve 20 comprises an inner expandable valve member 22 which supports flexible leaflets 24 therein. Typically, there are three leaflets 24 having peripheral cusp edges sewn to surrounding structure with free edges coapting against each other in the flow orifice through the valve member 22. As this is a replacement mitral valve, the stitched-in cusp edges 25 of the leaflets 24 are shown, with blood flow being only permitted down through the valve member 22 from the left atrium to the left ventricle LV. A similar arrangement is contemplated for the same prosthetic heart valve 20 implanted at the tricuspid annulus TA, with blood flow passing one way downward into the right ventricle RV.

A peripheral skirt 26 circumferentially surrounds the inner valve member 22, and a constricting suture 27 extends around a top edge of the skirt. The valve member 22 and skirt 26 comprise a fabric covering 28 sewn to a structural stent frame 30. An upper edge of the inner valve member 22 features a plurality of evenly spaced eyelets 32 formed by the stent frame 30. The eyelets 32 may be used to facilitate delivery of the expandable valve 20. For example, sutures or wires may be passed through the eyelets 32 to control the advancement and radial expansion/contraction of the valve.

With reference now to FIGS. 4A-4C, the stent frame 30 comprises a generally tubular inner portion 34 that defines the shape of the inner valve member 22. The eyelets 32 are shown at the top of a number of converging struts 36 that define the inner portion 34. In the illustrated embodiment, the struts 36 are formed in a generally serpentine pattern in the axial direction and secured to each other at nodes to form cells or openings therebetween. This structure is collapsible down to a minimum for delivery, and then expandable to the shape shown. It should be understood that the particular configuration of the struts 36 may be modified as is well known in the art. For example, another common pattern for the struts 36 defines a series of connected diamond-shapes which form similar shaped cells therebetween.

A plurality of outer arms 38 include generally arcuate posts 40 joined to the tubular inner portion 34 at lower bends 42. Each of the arms 38 features one or more outwardly-projecting barbs for anchoring in to surrounding tissue. In the illustrated embodiment, there are three barbs 44a, 44b, 44c extending outward from each of the posts 40. A first barb 44a angles downward, while the other two barbs 44b, 44c angle upward. Although the entire stent frame 30 will be covered with a biocompatible fabric, the barbs 44a, 44b, 44c project outwardly therethrough so that when the prosthetic valve 20 is expanded they embed in surrounding annulus tissue. As will be described below, a variety of different types of barbs are contemplated, and are generally interchangeable. The cloth-covered arms 38 define the peripheral skirt 26.

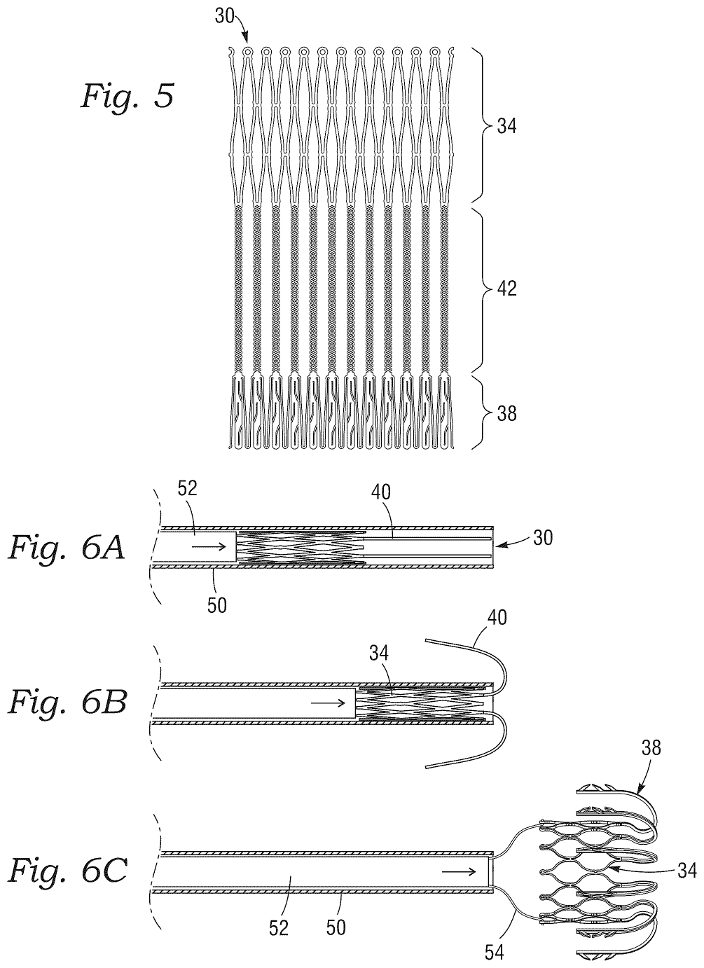

In the illustrated embodiment, as seen in the top plan view of FIG. 4C and in the laid-flat view of FIG. 5, there are 12 of the outer arms 38 evenly arrayed around the tubular inner portion 34. The 180° bends 42 connect each of the arms 38 to a pair of converging struts 36 at the lower end of the inner portion 34. The serpentine struts 36 extend upward and connect with two adjacent struts, finally converging with one of the adjacent struts to define the eyelets 32. There are thus 12 eyelets 32. Of course, the number of outer arms 38 and the strut configuration of the tubular inner portion 34 may vary, with a practical minimum of 6 and a practical maximum of 18.

The material of the stent frame 30 is such that it is capable of compression down to a small profile for passage through a delivery or access tube, while also being capable of expansion to the shape as shown which may be as large as 33 mm in diameter for the tubular inner portion 34. For self-expanding stent frames 30, the material is desirably a super elastic metal such as Nitinol. For balloon-expanding stent frames 30, the material is desirably stainless steel or a cobalt-chromium alloy such as Elgiloy. The struts 36 and posts 40 are shown with square or rectangular cross-sections, reflecting a common manufacturing technique of laser-cutting the stent frame from an initial tubular blank. Of course, round wires may alternatively be used to form the stent frame 30.

With reference back to FIGS. 3A and 3B, and also FIGS. 6A-6C, implantation and deployment of the prosthetic heart valve 20 will be described. FIGS. 6A-6C are sequential views showing a self-expandable valve stent 30 being expelled from an access tube 50 to illustrate steps in the deployment thereof. This valve stent 30 is shown without it cloth covering, and without any valve leaflets or other soft structure. The valve stent 30 defines the shape of the overall heart valve 20, and thus. Initially, a pusher 52 urges the valve stent 30 (i.e., heart valve 20) from a distal end of the access tube 50. The posts 40 that define the arms 38 are initially held by the access tube 50 in a linear, distally facing orientation. As they emerge from within the access tube 50, the arms 40 curl 180° in a proximal direction due to their inherent spring bias. Once again, a self-expanding stent frame 30 is desirably formed of Nitinol which can easily be shape set to assume the final configuration.

As the pusher two continues expelling the valve stent 30, the tubular inner portion 34 is eventually released and expands to its final diameter. FIG. 6C shows the valve stent 30 expanded to its maximum diameter. The outer arms 38 which form the fabric-covered peripheral skirt 26 contact the native valve leaflets first, and the barbs 44 embed themselves in the tissue. Tethers or wires 54 are shown extending from the valve stent 30 back into the access tube 50. Such tethers or wires can be used to manipulate the valves to third. For example, the wires may pass through the eyelets 32 at the proximal end of the tubular inner portion 34 and initially held tight so as to prevent the tubular inner portion from expanding once expelled from the tube 50. The tethers or wires 54 can then be slowly released to permit expansion of the inner portion 34. Conversely, if positioning of the heart valve 20 is incorrect, the tethers or wires 54 can be retracted within the access tube 50 to constrict the valve stent 32 allow for the position to be adjusted.

Now with reference back to FIG. 3B, the peripheral skirt 26 is shown being radially constricted. This can be accomplished by extending the constricting suture or cinch 27 around the top edge of the skirt 26. For example, a tunnel or pocket of fabric may be formed around the periphery of the top edge of the skirt 26 through which the suture or cinch 27 passes. By pulling on the cinch 27, the top edge of the skirt 26 is pulled inward closer to the inner valve member 22. By virtue of the barbs 44, the surrounding tissue comprising the valve annulus and leaflets is also pulled inward.

The process of cinching the surrounding tissue in this manner may be accomplished after closing the access incisions and under fluoroscopy. The sutures or cinch is 27 may extend out of the body through sealed incisions (i.e., with purse string sutures), typically through an elongated flexible tubular sheath (not shown). The constricting suture or cinch 27 is one example of an extrinsic actuator that can be used post-implant to reduce the overall diameter of the heart valve 20 which, because of the anchoring barbs 44, pulls the annulus inward simultaneously. Such an extrinsic constriction mechanism is in contrast with intrinsic constriction mechanisms disclosed elsewhere herein, as will become apparent.

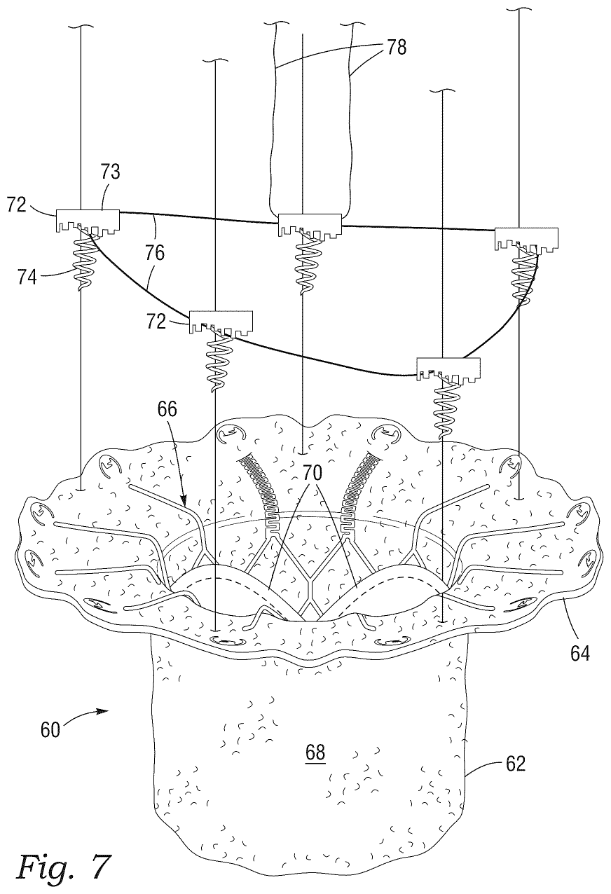

Now with reference to FIG. 7, an alternative expandable prosthetic heart valve 60 of the present application is shown having a generally cylindrical valve member 62 with an upper peripheral flange 64. The valve member 62 and peripheral flange 64 may be defined by a valve stent 66 covered with a biocompatible fabric 68. In the illustrated embodiment, the valve stent 66 has a plurality of connected struts to define the cylindrical valve member 62, as well as radial arms that extend outward at a proximal end of the valve member to define the peripheral flange 64. As described elsewhere, the valve stent 66 may be formed of a single self-expanding Nitinol stent, or maybe balloon-expandable as desired. As before, flexible leaflets 70 are sewn to the inside of the valve member 62 and define the one-way flow occluding surfaces. To reiterate, the prosthetic heart valve 60 is configured to be implanted at one of the atrioventricular valves.

A plurality of separate valve anchors 72 are shown exploded above the prosthetic heart valve 60 prior to deployment through the peripheral flange 64. The valve anchors 72 in this case are not integrated into the heart valve 60, and instead are separately deployed to ultimately form a part of the secured implant. The illustrated embodiment, each of the anchors 72 has an upper head 73 and a lower corkscrew-like tissue anchor 74. It should be understood that the particular configuration of the tissue anchor 74 may be other than a corkscrew, such as straight or curved barbs or the like. A lasso or cinch 76 passes through the head 73 of each of the anchor 72. Free ends 78 of the cinch extend proximally out of the body, preferably after passing through one of the anchor 72. Pulling on the free ends 78 reduces the circumference of the cinch 76.

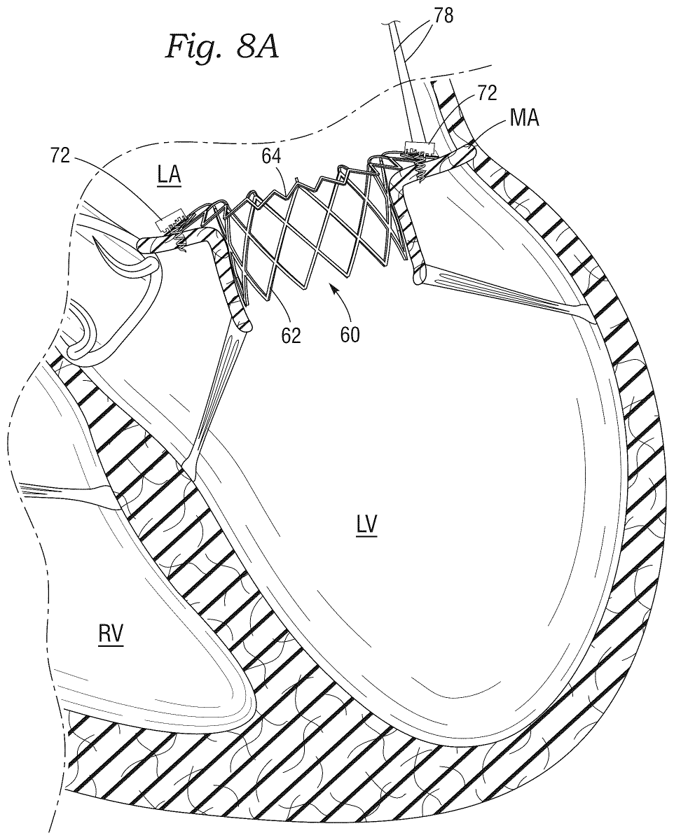

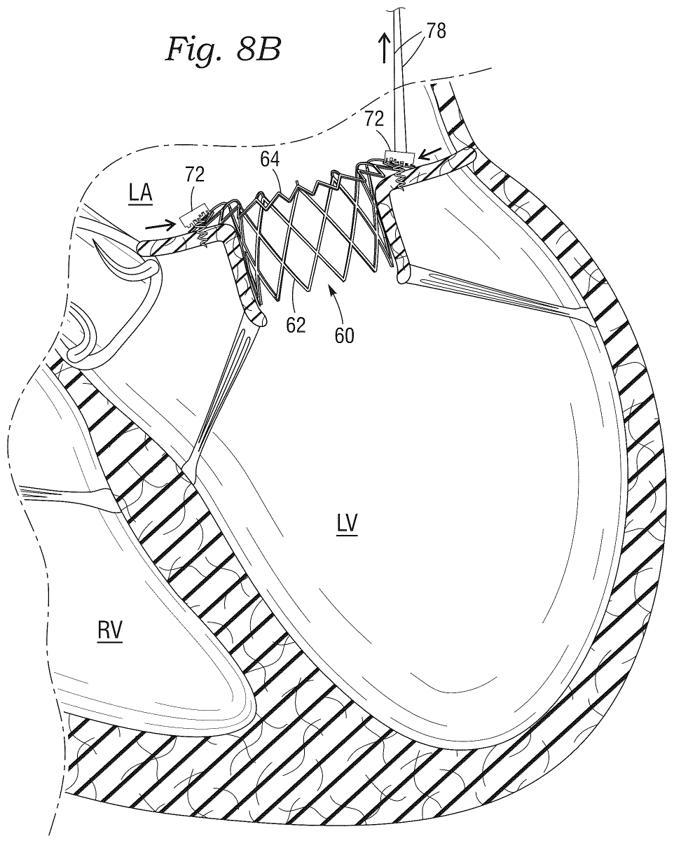

FIG. 8A is a sectional view through a left ventricle showing the expandable prosthetic heart valve 60 of FIG. 7 prior to a final step in deployment within the mitral annulus. The valve stent 66 has been expanded such that the valve member 62 is in contact with the leaflets of the mitral valve. The anchors 72 have been deployed around the peripheral stent 64 such that they are embedded in the mitral annulus MA. The free ends 78 of the cinch 76 pass out of the body, typically through a tubular sheath (not shown). FIG. 8B is the same view after a final step of radially constricting the peripheral flange 64 anchored to the mitral annulus MA by pulling on the free ends 78 of the cinch 76. This pulls the anchor 72 inward which, in turn, pulls the surrounding tissue leaflets and annulus into better contact with the exterior of the valve member 62. Once again, FIGS. 8A and 8B show just the valve stent 66 for better clarity, but it will be understood that the fabric covering 68 and leaflets are part of the finished valve 60. Here again, the cinch 76 acts as an extrinsic constriction mechanism that is actuated from outside the body, or at least is not intrinsic to or carried by the heart valve itself.

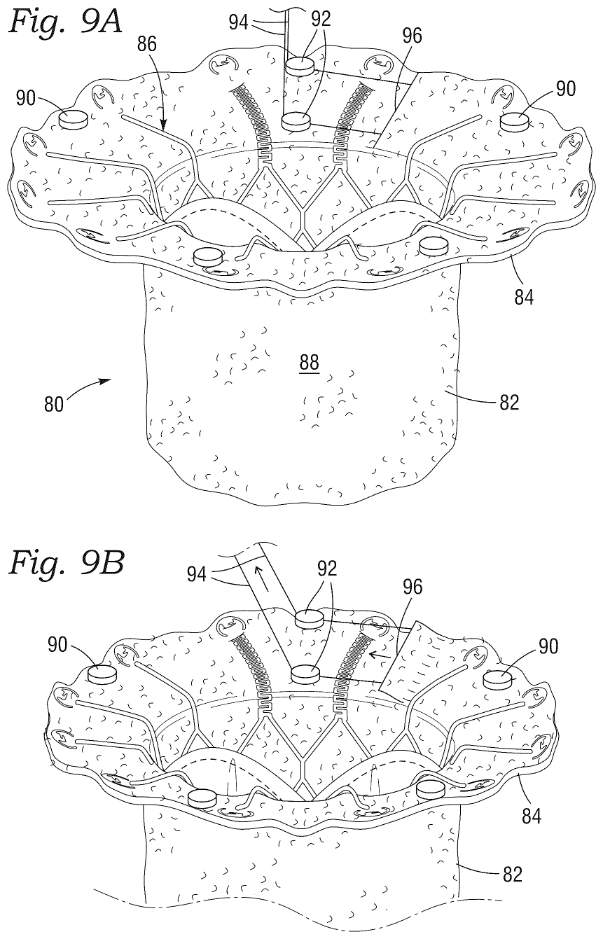

FIG. 9A is a perspective view of a still further expandable prosthetic heart valve 80 having a valve portion 82 and an upper peripheral flange 84, similar to the valve shown in FIG. 7. The valve 80 may again be formed by a structural valve stent 86 covered with fabric 88.

A plurality of anchors 90 are shown deployed around the peripheral stent 84. The anchors 90 may be as described above, such that just the heads are shown with a corkscrew or otherwise tissue piercing member (not shown) embedded through the flange 84 into annulus tissue. Two of the anchors 92 are radially spaced from one another at a location around the peripheral stent 84 and each has a tether 94 extending therethrough. The tethers 94 pass through the anchors 92 and extend circumferentially through or on top of the peripheral flange 84 to be connected to a free edge 96 thereof. The peripheral flange 84 is discontinuous at the free edge 96 such that it has a variable circumferential profile. FIG. 9B shows a final step of circumferentially cinching the peripheral flange 84 to cause radial constriction thereof by pulling on the tethers 94. The free edge 96 is pulled towards the anchors 92, which cinches the peripheral flange 84. By virtue of a reduction in the circumferential perimeter of the flange 84, surrounding tissue is pulled inward toward the valve member 82.

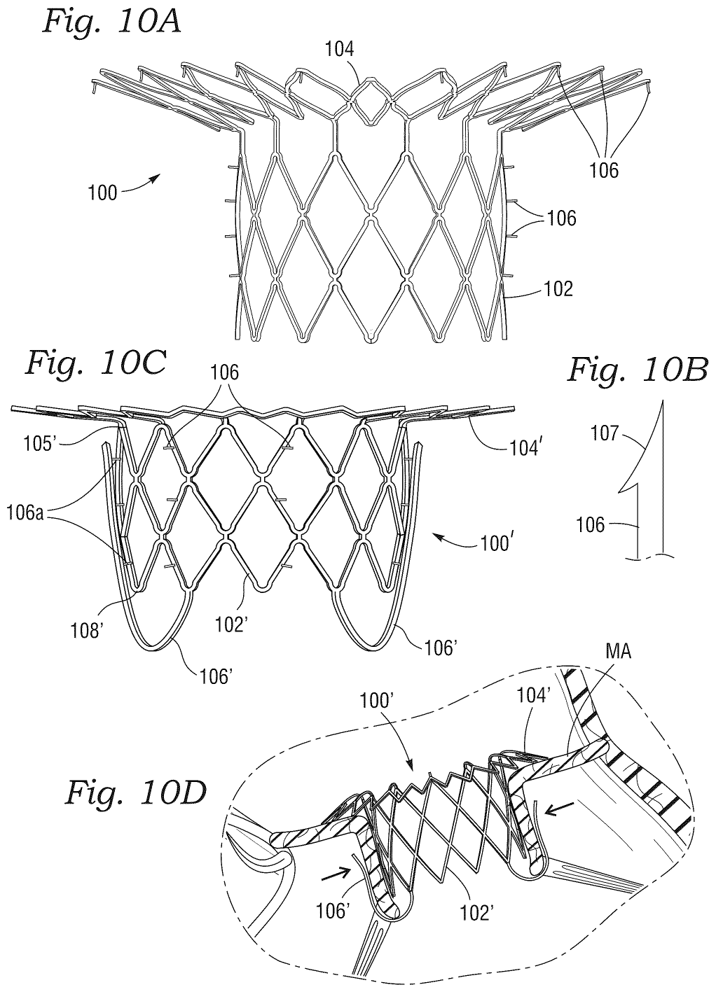

FIG. 10A is an elevational view of an alternative heart valve stent 100 having a tubular valve portion 102 and an upper peripheral flange 104 with anchoring barbs 106. Anchoring barbs 106 may also be provided extending radially outward from the valve portion 102, as seen. In this embodiment, the struts of the valve stent 100 are formed in diamond shapes, with the barbs 106 defined by a sharp points. As seen elsewhere in this application, the stent 100 is shown without an outer fabric cover and valve leaflets. The assembled heart valve may be formed by the single expandable super-elastic stent 100 covered with fabric, and flexible valve leaflets are supported within the valve portion 102 to enable one-way blood flow through the valve (downward for this mitral valve).

FIG. 10B shows one example of an anchoring barb 106. The barb 106 may include a shaft that extends perpendicularly or at an angle from the particular host structure (flange, main body, etc.), with a small hook 107 or other such device near the end to help prevent the barb from pulling free from tissue. There may be multiple such hooks 107.

FIG. 10C shows a similar valve stent 100′ that comprises a main body 102′ and an upper peripheral flange 104′ that extends radially outward from the upper end 105′ of the main body 102′. In this embodiment, the upper peripheral flange 104′ extends substantially perpendicular to the stent opening defined by the upper end 105′. Ventricular anchors 106′ curl down and back up about 180° from a lower end 108′ of the stent main body 102′. The ventricular anchors 106′ are shaped for capturing a native valve leaflet between the ventricular anchor and the main body 102′ of the stent. In conjunction with or in substitution of constricting barbs on the upper peripheral flange 104′, the ventricular anchors 106′ may be configured to constrict post-implant, such as by providing a cinch around the periphery thereof, such as in the embodiment of FIGS. 7-8. The disk-like upper peripheral flange 104′ can be positioned flat across the top surface of the mitral annulus MA and provide increased surface area contact for tissue ingrowth.

The main body 102′ may have outward barbs 106, much like the stent 100 in FIG. 10A. Additionally, inward barbs 106a may be provided along each of the ventricular anchors 106′, with the barbs being like those shown and described with respect to FIG. 10B.

FIG. 10D shows the valve stent 100′ implanted at a mitral annulus MA. The ventricular anchors 106′ deploy to the outside of the mitral leaflets and are configured to constrict inward, as shown. This helps secure the valve having the stent 100′ within the annulus.

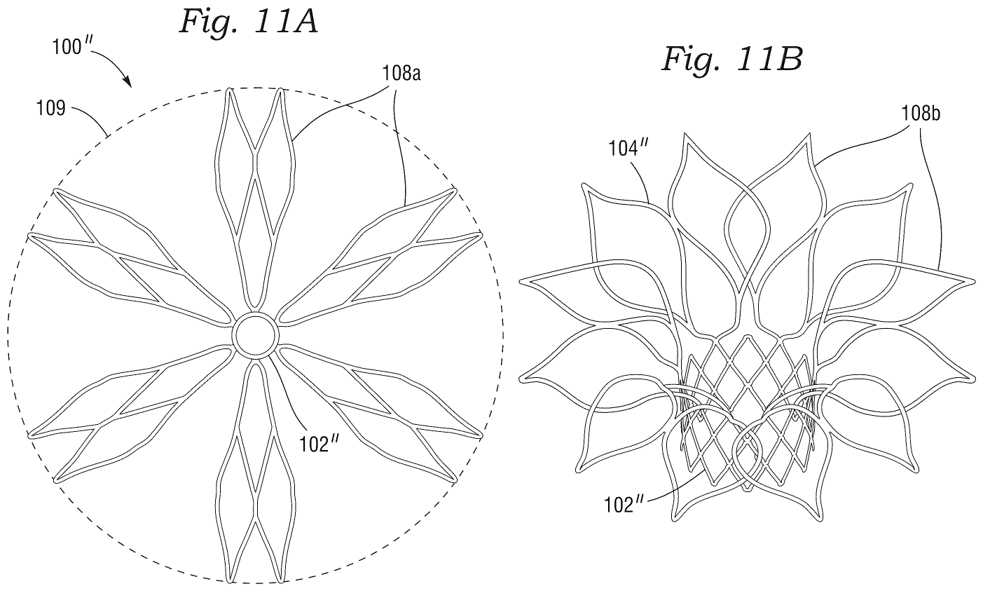

FIGS. 11A and 11B are two views illustrating a step in the deployment of a valve stent 100″ like that in FIG. 10A wherein expansion of the central valve portion 102″ causes radial constriction of the upper peripheral flange 104″. Namely, radial expansion of the valve portion 102″ causes the petal-shaped struts 108a of the peripheral flange 104″ to circumferentially expand (as seen at 108b) which caused their outer tips to radially constrict from an initial diameter seen at 109. Since the barbs on the struts 108a are already embedded in surrounding annulus tissue, this pulls the tissue toward the valve. This is an example of an intrinsic constriction mechanism, in that the radial constriction of the valve and surrounding annulus are caused by structures carried by or intrinsic to the heart valve, as opposed to requiring an external constrictor.

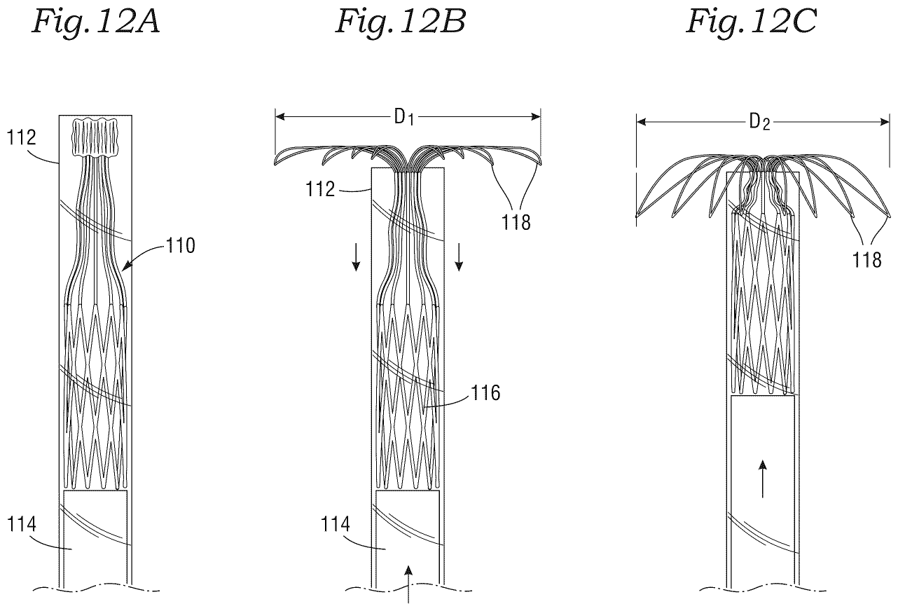

FIGS. 12A-12C are sequential views showing a still further valve stent 110 being expelled from an access tube 112 to illustrate steps in the deployment thereof. As before, a pusher 114 may be used to advance the valve stent 110 from within the tube 112, potentially in conjunction with retraction of the tube as shown in FIG. 12B. Initially, a tubular valve portion 160 remains within the access 112, while a peripheral flange 118 expands radially to a first diameter D₁. Upon further expulsion from within the tube 112, the peripheral flange 118 curls back upon the access tube 112 and in the process the radially outer tips constrict inward to a second diameter D₂ smaller than D₁. As with the embodiment shown in FIG. 10, the tips of the peripheral flange 118 have barbs that embedded within the annulus tissue such that this reduction in diameter pulls the tissue inward around the sequentially expanded valve portion 116. Again, this is an intrinsic constriction mechanism as the radial force is generated by the changing shape of the valve stent 110 itself.

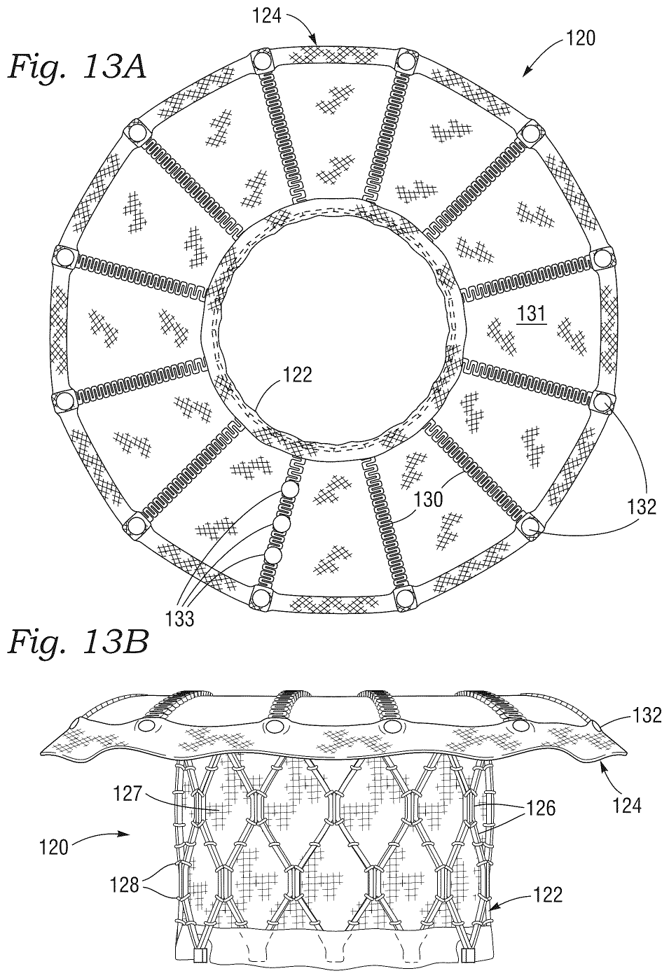

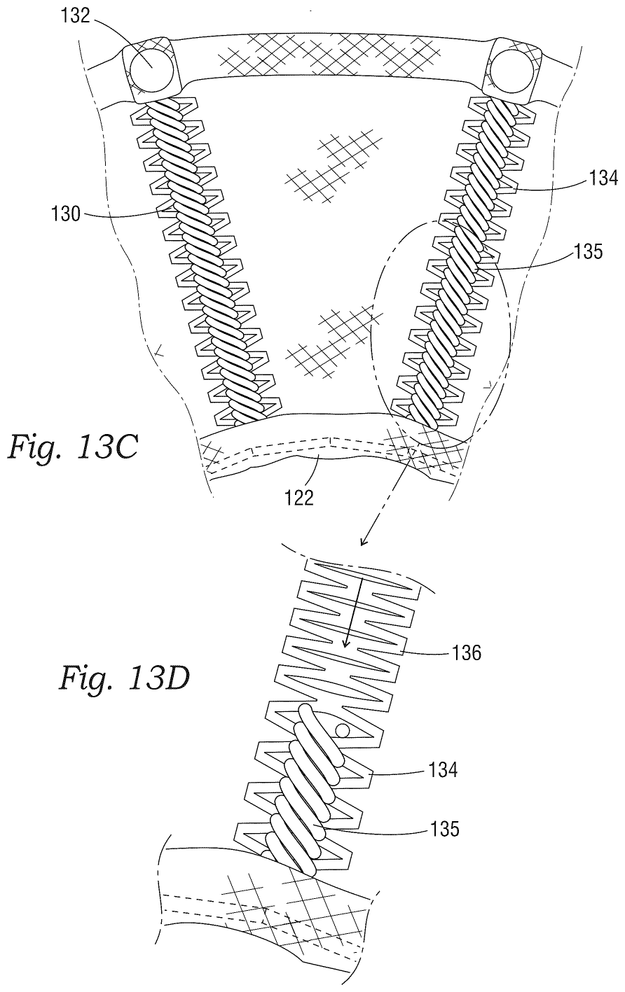

FIGS. 13A and 13B are top plan and side elevational views of an exemplary expanded prosthetic heart valve 120 defining a tubular valve portion 122 having a peripheral stent 124. As described elsewhere, the valve 120 may be defined by an expandable valve stent 126 covered with fabric 127 via a plurality of connecting sutures or stitches 128. FIG. 13A shows radial arms 130 of the valve stent 126 that extend outwardly from an inlet (proximal) end of the valve portion 122 and, along with a fabric skirt 131, define the peripheral flange 124. One or more tissue anchors 132 may be secured to each radial arm 130, or the outer ends may be reinforced to receive a separately deployed anchor, such as was described above with respect to anchors 72 shown in FIG. 7. Anchors are illustrated at the ends of each radial arm; however, anchors may be included along the length of each arm 130, such as illustrated by anchors 133 in one arm. The anchors 132, 133 may be as described elsewhere in the present application. This particular configuration of a heart valve 120 may be seen as a model for a number of embodiments described herein, in which a fabric covered peripheral flange at the proximal end of a fabric cover valve member provides the tissue constricting structures in the form of a variety of constricting arms connected by fabric. Although the flange is illustrating having a particular shape, it should be understood that the flange could have different shapes and is preferably conformable to the shape of the surrounding tissue, such as along the top of the annulus.

FIG. 13C is an enlarged portion of FIG. 13A showing details of the peripheral flange 124 and one particular embodiment of constricting arm 130. Each of the arms 130 comprises an elongated series of diamond-shaped struts 134 connected in series and extending from the inner valve portion 122 to each anchor 132. The diamond-shaped struts 134 form an extended spring biased to a constricted length and held extended. Namely, a filament or suture 135 coils along and through the diamond-shaped struts 134. The struts 134 are held in tension by the suture 135, so that they are radially expanded when the heart valve 120 is implanted at the annulus using the anchors 132.

FIG. 13D is an enlarged portion thereof showing a constricting mechanism in each arm 130. Namely, the suture 135 is biodegradable in the body after a period of time. The upper portion shows the suture 135 dissolved, while some remain close to the inner valve portion 122. The struts 136 in the portion without the coiled suture 135 have collapsed such that the diamond shaped openings are now much smaller due to the resiliency of the struts. The inward radial arrow indicates the overall constriction of the arm 130, which pulls the anchor 132 inwardly, thus cinching the surrounding annulus tissue toward the inner valve portion 122. The rate of degradation of the sutures 135 may be regulated so that the inward cinching occurs rapidly, as in a few days, to more slowly, such as in a month or two, depending on the needs. Here again we see a slightly different intrinsic constriction mechanism where the radial force is generated by disintegration of the suture 135 and reduction in length of the constricting arms 130.

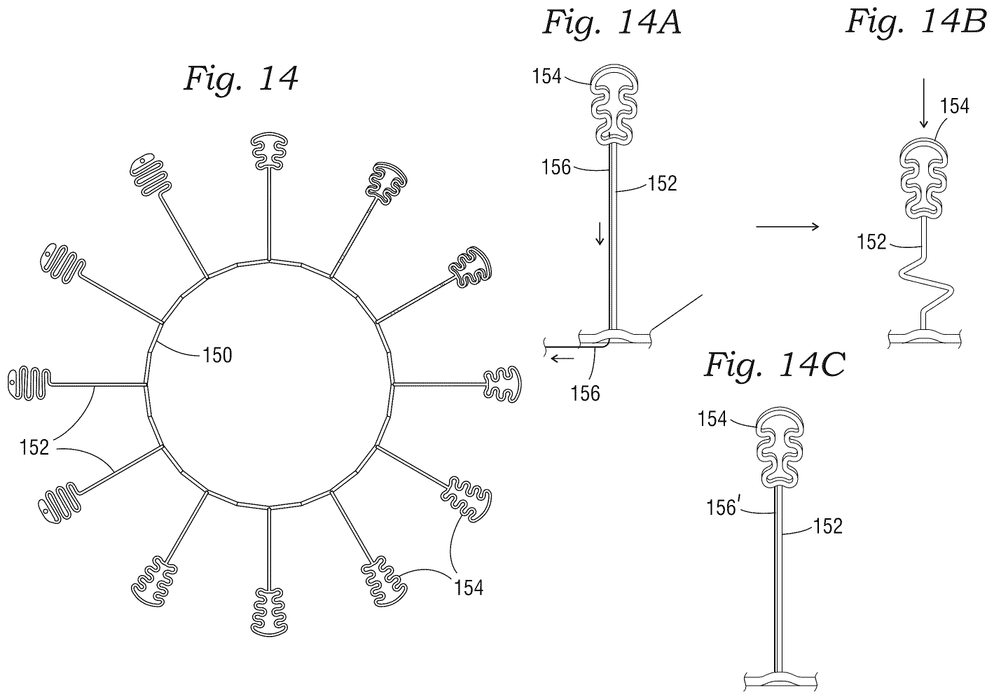

FIG. 14 is a top plan view of an upper peripheral flange of an exemplary valve stent 150 of the present application defined by a plurality of radial arms 152 having anchors 154 on their outer ends. As seen best in FIGS. 14A and 14B, actuation of one radial arm before and after radial constriction includes removing a stiffening wire or member 156 so that the arm 152 collapses. More particularly, the arms 152 are desirably shape set into the radially constricted shape and maintained in their linear configuration by the stiffening member 156. The shape set arms 152 form an extended spring biased to a constricted length and held extended by the stiffening member 156. The stiffening member 156 may be a generally flexible wire having sufficient column stiffness to resist collapse of the arms 152. The stiffening members 156 desirably extend along the arms 152 and through a hole formed in the anchor 154. Each of the stiffening members 156 releases each of the arms 152 to collapse. It should be understood that the valve is first expanded and the anchors 154 embedded in tissue prior to pulling all of the stiffening members 156 free, at which point the arms 152 and peripheral flange defined thereby radially collapses. This is a hybrid version of constricting mechanism which requires removal of an external element, the stiffening members 156, but radial constriction is then generated by the reduction in length of the radial arms 152 which is intrinsic to the heart valve. A hybrid constricting mechanism thus requires an external “triggering” action, but the source of the constricting force is part of, or carried by the heart valve and remains implanted. If, conversely, the radial arms 152 were configured to be bioresorbable, the constricting mechanism would be entirely intrinsic to the heart valve.

Another intrinsic example of the same constricting mechanism is shown in FIG. 14C, where the stiffening member 156′ is a rod or stick carried by the stent 150 and configured to dissolve after a period of time. For instance, the stiffening member 156′ may resorb within several minutes after exposure to blood, which provides enough time to securely embed the tissue anchors before the radial arms 152 collapse and the flange pulls the surrounding tissue inward.

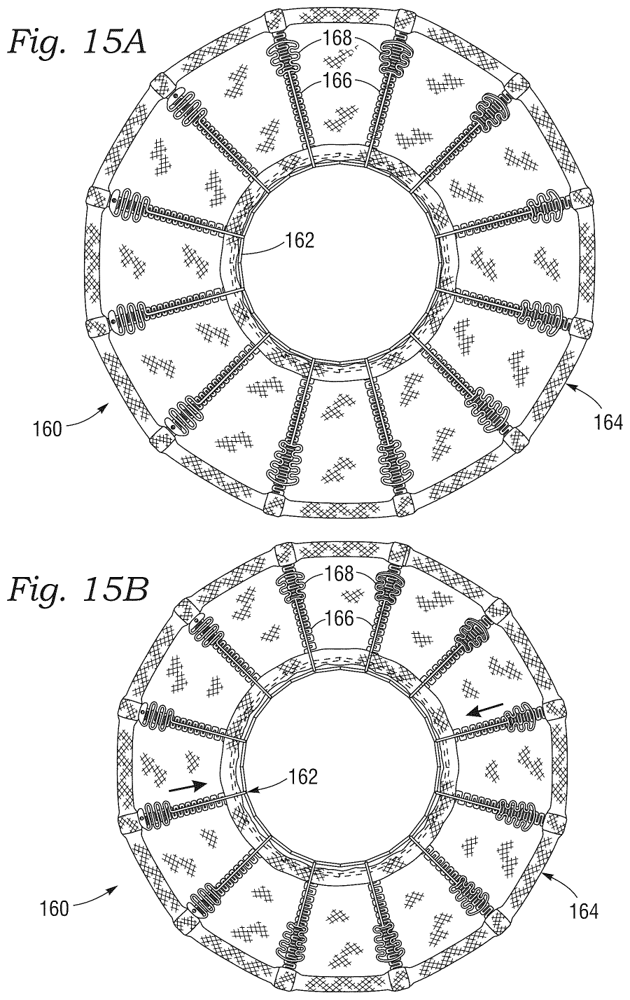

FIG. 15A is a top plan view of an exemplary prosthetic heart valve 160 with a central valve member 162 and a peripheral flange 164 similar in construction to that of FIG. 13A. An inner stent frame has a peripheral flange like that shown in FIG. 14. Namely, flange 164 has arms 166 that terminate at outer anchors 168. The arms 166 are radially collapsible and the anchors 168 have barbs or may be secured to annulus tissue with separate anchors. When desired, the arms 166 may be collapsed as shown in FIG. 15B causing radial constriction of the peripheral flange 164. The anchors 168 pull the surrounding tissue inward against the central tubular valve member 162, thus ensuring a tighter fit. As described herein, arms 166 such as shown may be configured to collapse in a variety of ways, and likewise, the fabric-covered valve structure shown in FIG. 15A may be adapted in various ways. The radial arms 166 may provide an intrinsic constricting mechanism or may be a hybrid extrinsic/intrinsic type if an external release wire or suture is used.

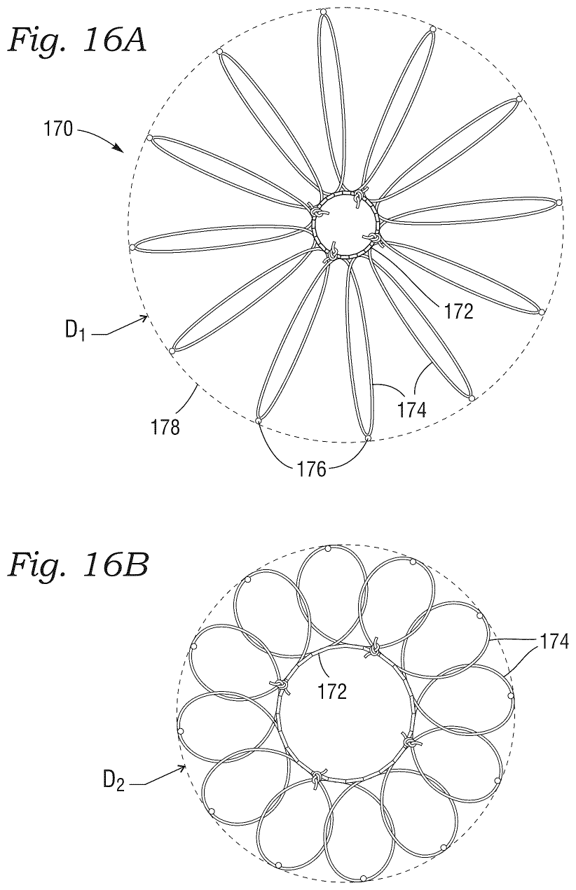

For an entirely intrinsic example, FIGS. 16A and 16B are top plan views of another exemplary valve stent 170 having a central valve portion 172 and radially constricting peripheral flange with a series of outer petals 174. Anchors or eyelets 176 as shown are provided on the outer ends of the petals 174 and the stent structure is fabric covered as explained herein. After expansion at the implantation site, as in FIG. 16A, the peripheral flange has a diameter D₁ defined by a circle of revolution 178 of the anchors 176. Subsequently, the barbs on the anchors 176 or separate barbs through the anchors 176 are deployed into the annulus tissue. FIG. 16B shows the valve stent 170 after radial constriction. The petals 174 are preferably super-elastic and expand circumferentially when the central valve portion 172 radially expands. This constricts the outer ends of the petals 174 to a circle of revolution having a diameter D₂ and thus pulls the surrounding tissue inward.

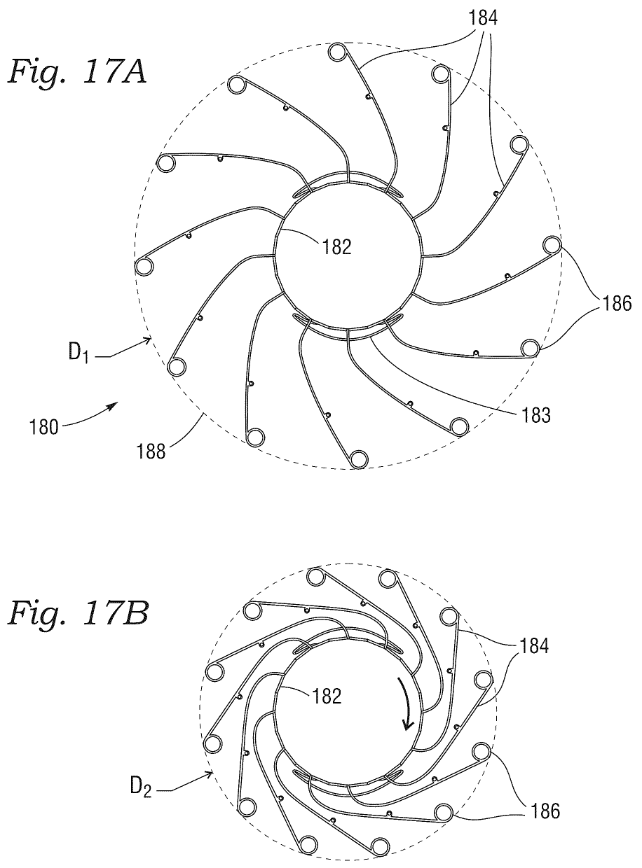

FIGS. 17A and 17B are top plan views of another exemplary valve stent 180 having a central valve portion 182 and radially constricting peripheral flange defined by a series of spirally arranged arms 184. The stent 180 may also have two or more ventricular anchors or arms 183 that are positioned to expand around the outside of the valve leaflets, much like the ventricular anchors 106′ seen in FIG. 10C. The arms 183 curl down and back up about 180° from a lower or outflow end of the stent main body. Again, outer ends of the arms 184 have anchors or eyelets 186 that when expanded together define a circle of revolution 188 having a diameter D₁. After expansion of the valve and securing the anchors or eyelets 186 to surrounding tissue, the central valve portion 182 is rotated about its own axis to pull the anchors or eyelets 186 inward. That is, as the valve portion 182 rotates the inner ends of the arms 184 they pull in the outer ends to a constricted diameter D₂. The rotation of the valve portion 182 must be initiated externally, but the radial constriction of the heart valve is generated by an intrinsic mechanism. Tus, this is a hybrid version of constricting mechanism.

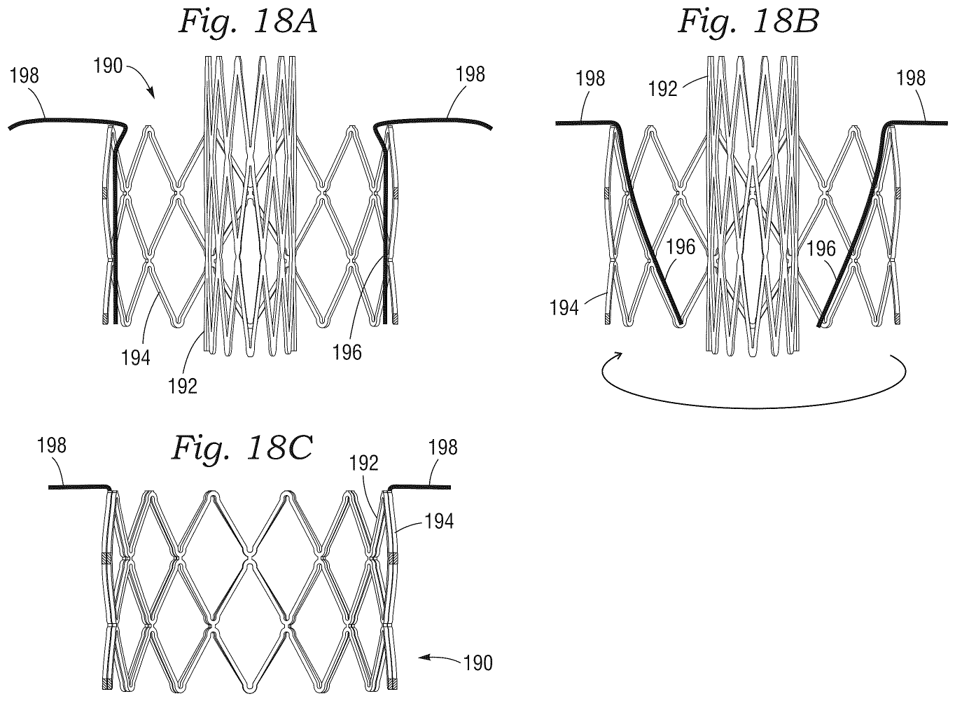

In a slightly different configuration, FIGS. 18A-18C are schematic elevational views of a two-part expandable prosthetic heart valve stent assembly 190 showing several steps in deployment thereof. The assembly 190 includes an inner cylindrical valve member 192 that ultimately expands outward into contact with a surrounding anchor stent 194. The stent 194 has a plurality of struts 196 built therein with radial arms 198. Although not shown, the arms 198 each have barbs at their distal ends.

Initially, the anchor stent 194 is advanced through an access tube into and implantation position within one of the atrioventricular valves. Expansion of the anchor stent 194 is accomplished at the same time that the radial arms 198 embed in surrounding tissue. The inner valve member 192 is positioned within the anchor stent 194 but remains unexpanded. Subsequently, as indicated in FIG. 18B, the anchor stent 194 is rotated about its axis so that the struts 196, being connected at a lower end of the anchor stent, rotate into helical shapes. Because the outer arms 198 are anchored to tissue, this tends to pull the tissue inward. Finally, the inner valve member 192 is expanded as seen in FIG. 18C. Because the anchor stent 194 has pulled in the surrounding tissue, the fit of the valve member 192 is improved. Again, this is a hybrid constriction mechanism that requires external input to rotate the anchor stent 194, but ultimately the radial constriction is generated by an intrinsic mechanism built into the heart valve.

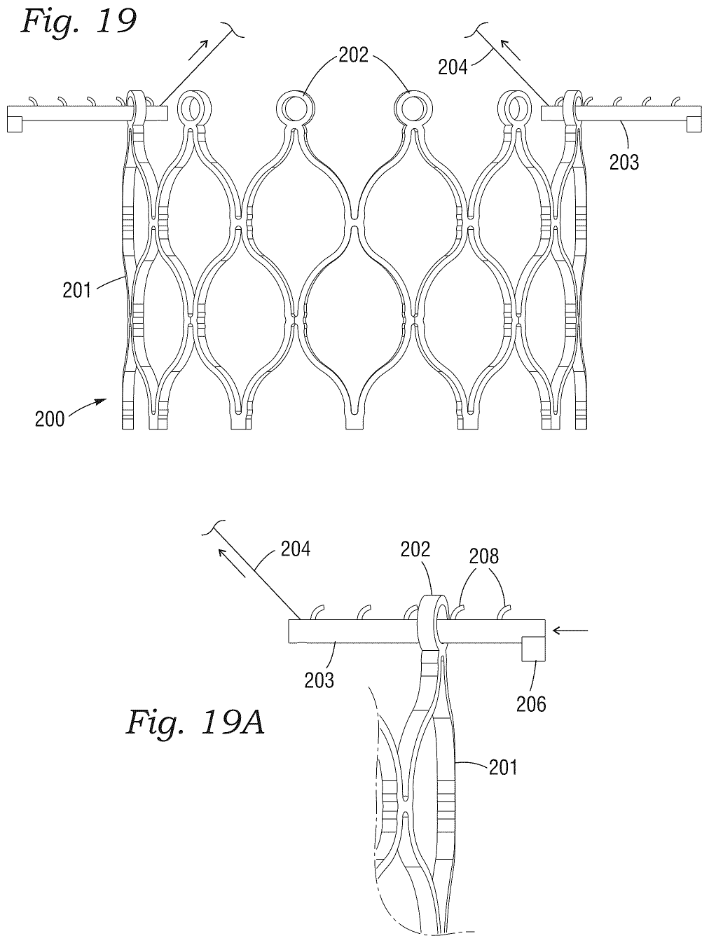

FIG. 19 is elevational view of another valve stent 200 having a central valve portion 201 with upper eyelets 202. Radially retractable arms 203 distributed around the upper periphery and through the eyelets 202 form a peripheral flange. Although only two arms 203 are shown, the stent 200 may have 6 or up to 18 such arms. Each arm 203 is connected to an externally actuated tether 204 that is configured to pull the arm radially inward, and a barb 206 on the outer ends of the arms embeds in annulus tissue. FIG. 19A illustrates inward retraction of one of the arms utilizing a ratcheting mechanism 208 to prevent outward movement. The tethers 204 may be simultaneously pulled to constrict the peripheral flange formed by the aggregate arms 203, thus constricting the annulus around the central valve portion 201.

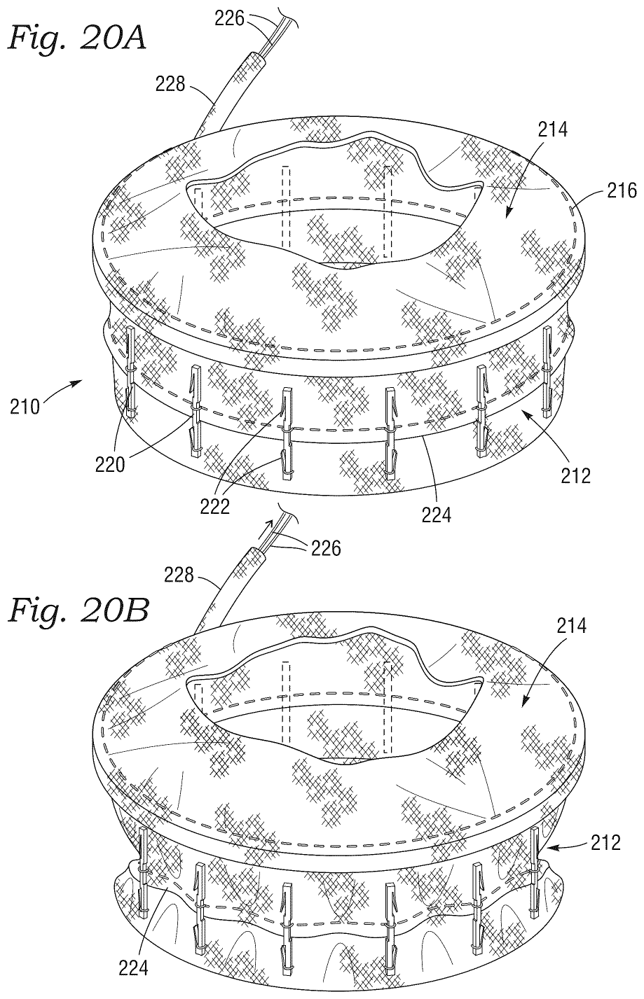

FIGS. 20A and 20B are perspective views of an outer fabric structure 210 useful in constriction of expandable prosthetic heart valves in both initially deployed and constricted configurations, respectively. The fabric structure 210 comprises a generally tubular outer wall 212 having an upper flexible skirt 214 secured thereto by a line of stitching 216. Although not shown, the fabric structure 210 may comprise an inner super-elastic skeleton that defines its overall cylindrical shape but which is easily collapsed for delivery and later during constriction of the structure.

The outer wall 212 has a series of axial or vertical struts 220 on its exterior, each of the struts featuring one or more outwardly-projecting barbs 222. The struts 220 span an annular horizontal junction in the wall 212 that forms a circular pocket 224 for a pair of flexible cinching elements 226. The cinching elements 226 passed through a tubular sheath 228 before entering the pocket 224 and diverging around the circumference of the wall 212. Pulling on the cinching elements 226 from an external location thus enables constriction of the tubular wall 212, as seen in FIG. 20B.

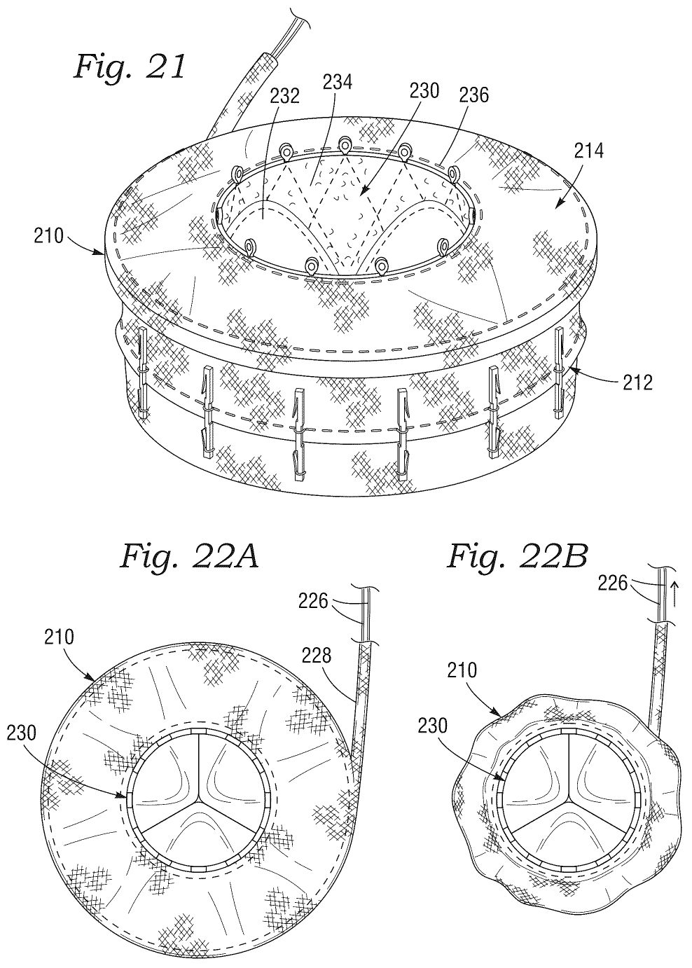

FIG. 21 is a perspective view of an assembly of the outer fabric structure 210 of FIG. 20A with a prosthetic heart valve 230 mounted therein. The heart valve 230 may be similar to those described above, with flexible leaflets 232 sewn within a tubular fabric-covered valve stent 234. An inner circular orifice of the flexible skirt 214 may be secured to a top edge of the heart valve 230 via stitching 236.

FIGS. 22A and 22B are top plan views of the assembly of FIG. 21 before and after radial constriction of the outer fabric structure 210. More particularly, after delivery of the assembly into position within the target annulus, both the fabric structure 210 and the heart valve 230 are expanded. Consequently, the barbs on the outer wall 212 of the fabric structure 210 engage annulus tissue. Before or after closing up the operating site, the cinching elements 226 are pulled to reduce the size of the fabric structure 210, as seen in FIG. 22B. This pulls the surrounding annulus inward toward the cylindrical heart valve 230, thus improving contact therebetween. This is another extrinsic constriction mechanism.

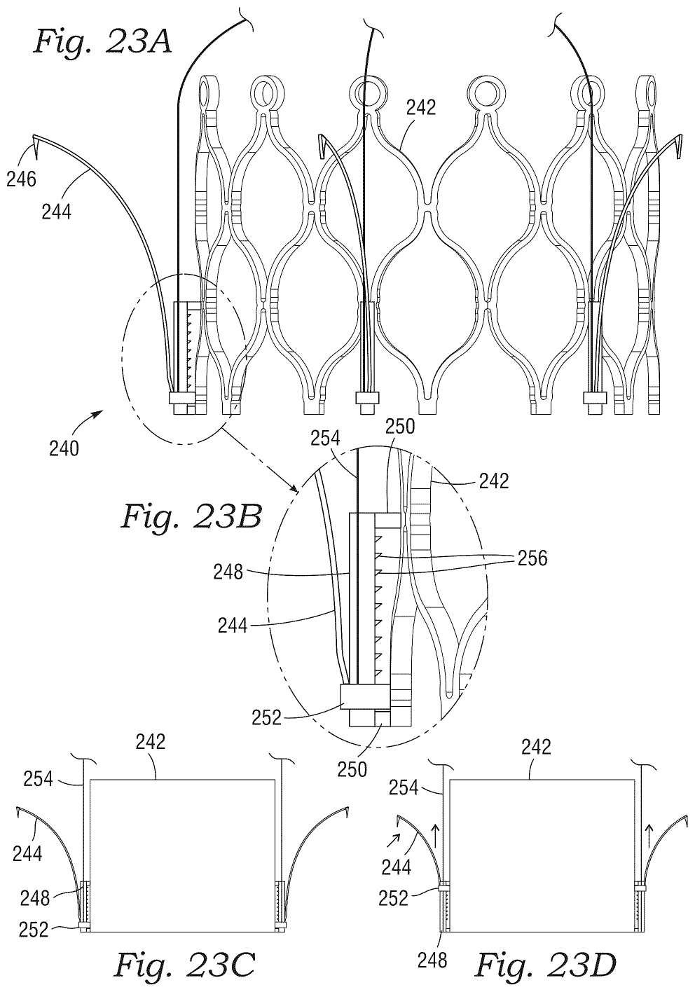

FIG. 23A is elevational view of another valve stent 240 having a tubular valve portion 242 and a plurality of outer radially retractable arms 244. Each of the arms 244 couples to and is cantilevered from a lower end of the valve portion 242 and has a barb 246 on a terminal extremity thereof. With reference to the enlargement of FIG. 23B, a retraction mechanism for each of the arms 244 is described. Namely, each arm 244 is secured to an axial post 248 fixed at a lower end of the valve portion 242 by, for example, braces 250. A collar 252 is arranged to travel vertically upward along the post 248 when pulled by an external tether 254. The collar 252 is limited to upward movement by a series of ratchet teeth 256 provided on the axial post 248.

FIGS. 23C and 23D are schematic views showing operation of the constricting arms 244. Specifically, pulling upward on the external tethers 254 lifts the collars 252 along each of the posts 248. Because the arms 244 are biased at a gentle outward arc, the collars 252 cam each of the arms radially inward. Separation is done after expansion of the heart valve within the annulus so that the barbs 246 on each of the arms 244 engages surrounding tissue. Under fluoroscopy, the desired amount of constriction of the surrounding tissue may be determined. Once again, this enhances the engagement between the surrounding tissue and the prosthetic heart valve.

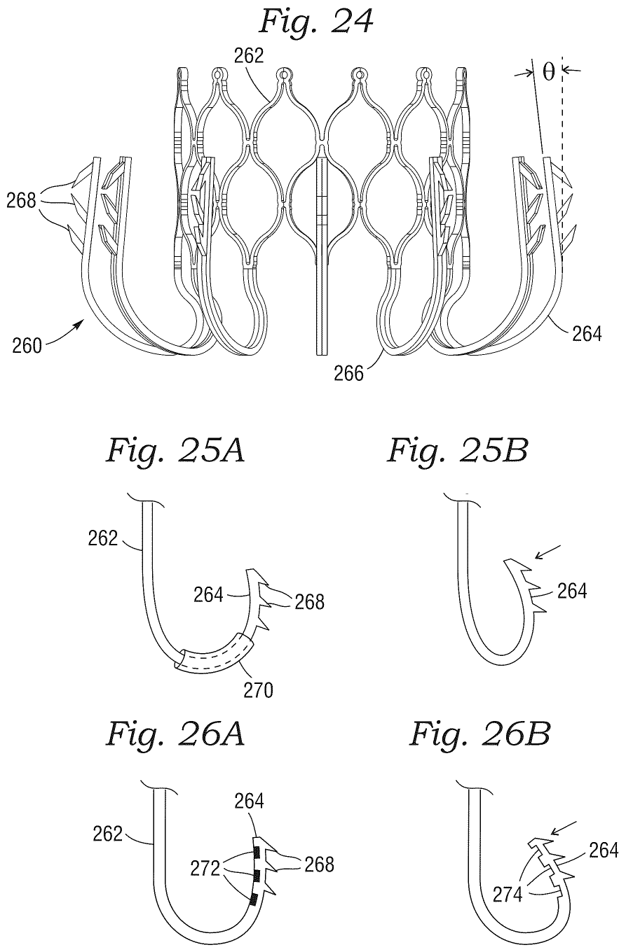

FIG. 24 is elevational view of yet another valve stent 260 with a central tubular valve portion 262 connected to a plurality of outer, generally axially oriented arms 264 via lower U-bends 266. Each of the arms 264 has outer barbs 268 thereon and is biased slightly inward toward the valve portion 262. That is, each of the arms 264 has an inward bias that creates a slight inward angle θ from the vertical. The angle θ may be between 5-20°.

FIGS. 25A and 25B are views of one of the arms 264 of the valve stent 260 before and after dissolution of a temporary measure to hold the arms outward. More particularly, each of the arms 264 initially has a stiffening tube 270 mounted thereon, preferably at the lower U-bend 266. The stiffening tubes 270 have a greater radius of curvature than the U-bends 266 to force the arms 264 into a more vertical orientation. Each of the tubes 270 is bioresorbable after a certain time within the body, and subsequently, as seen in FIG. 25B, the arms 264 revert to their initial inward angle. In one embodiment, the stiffening tubes 270 resort after a period between one week and a month within the body, which serves to pull the surrounding tissue inward by virtue of the engagement of the barbs 268. This is wholly intrinsic to the heart valve.

FIGS. 26A and 26B are views of one of the arms 264 before and after dissolution of another temporary measure to hold the arms outward. In this version, a number of sitting stiffening plugs 272 are initially positioned within recesses 274 on an inner side of the arms 264. The stiffening plugs 272 also are bioresorbable such that after they dissolve, the arms 264 are once again biased inward to the relaxed orientation. Those of skill in the art will understand that there are various ways to produce such a delayed inward movement of the arms 264.

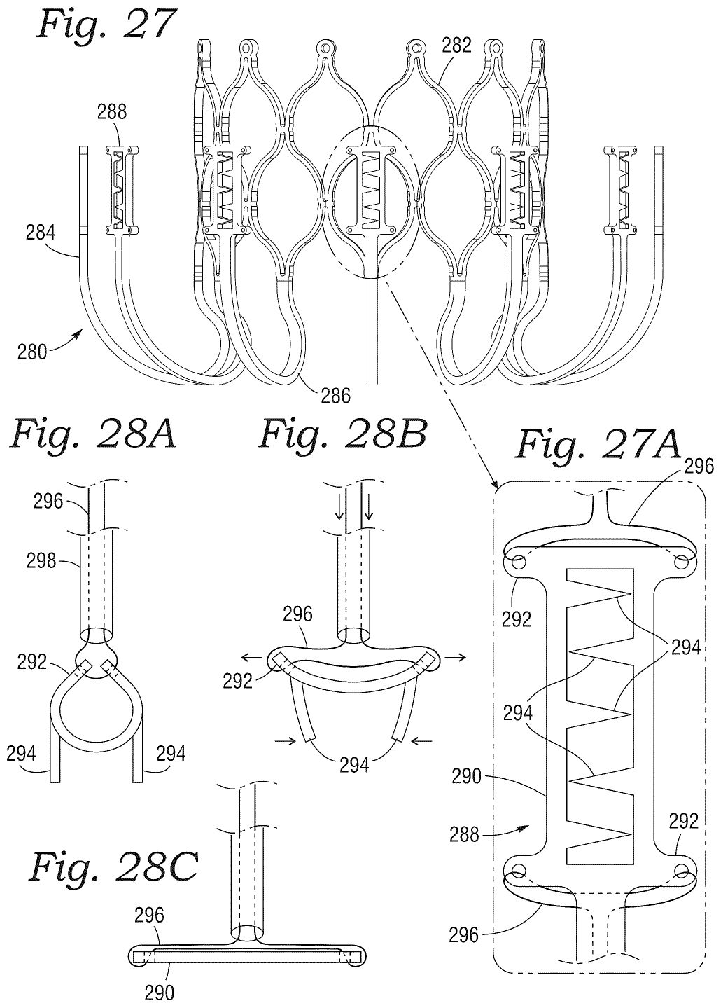

FIG. 27 is elevational view of still another valve stent 280 having an inner tubular valve portion 282 connected to a plurality of axially oriented outer arms 284 by lower U-bends 286. Each of the arms 284 has an active tissue anchor 288 at its distal end. An “active” tissue anchor in this sense means one that can be manipulated to grab tissue as a barb-type anchor which is simply pressed against tissue to engage.

FIG. 27A is an enlargement of one of the active tissue anchors 288, which primarily includes a generally rectangular frame 290 having oppositely-projecting levers 292 on each end. The rectangular frame 290 defines a central opening into a plurality of teeth 294 project. The tissue anchor 288 may be formed of super-elastic material, and has a flat relaxed configuration, as seen in the top view of FIG. 28C.

It should be noted that the orientation of the clamping anchor 288 can vary from the hinging/flexing levers 292 on top and bottom with teeth 294 oriented inward from left and right sides as shown, or perpendicular with the hinging/flexing levers 292 instead on left and right sides and teeth 294 oriented inward from top and bottom sides. Also, the location that any of the levers 292 attach to the frame may be relocated to low strain areas so as not to impede the hinging/flexing sides of the anchor 288. That is, rather than locating the lower hinging/flexing levers 292 at the connection to the U-bends 286, the anchor 288 may be oriented horizontally to decouple the flexing of the levers from the flexing of the U-bends 286.

FIGS. 28A-28C are top plan views of one of the active tissue anchors 288 showing several phases of deployment. Initially, external tension members 296 are used to hold the tissue anchors 288 in a tensed configuration, seen in FIG. 28A. Namely, the tension members 296 comprise flexible filaments or sutures which pass through apertures in each of the outwardly-projecting levers 292. By pulling on the levers 292 through the use of an outer sheath 298, the frames 290 are bent back upon themselves so that the teeth 294 project radially outward. Once the heart valve has been introduced within the annulus and expanded, the outwardly-projecting teeth 294 pierce the surrounding tissue. Subsequently, the tension members 296 are relaxed, as seen in FIG. 28B, which permits the frames 292 two spring backward toward their flat, relaxed configuration, which in turn pivots the teeth 294 inward toward each other. This action causes the tissue to be grabbed firmly between the jaws, much like a bear trap.

The active tissue anchors 288 may be utilized with any of the various valve stent embodiments described herein in place of barbs, for example. After grabbing the tissue with the active tissue anchors 288, a cinch mechanism is deployed to pull the tissue inward toward the central valve portion 282. The cinch mechanism may be any of those described herein.

The present application presents a number of expandable prosthetic heart valves configured to constrict the target annulus at the time of or after implant to enhance the engagement therewith. A number of embodiments contemplate manipulating components of the heart valve from outside the body once the valve has been implanted. This technique involves snaking sutures or other such control elements from the target annulus through sealed incisions to the exterior of the body. In the same manner, guide sutures pre-installed around the target annulus may be used to control and/or steer the heart valve as it is being advanced to the annulus. An additional technique more commonly used with surgical heart valves implanted using open heart surgery involves pre-installing tissue anchors around the annulus and then parachuting the expandable heart valve down an array of sutures coupled to the tissue anchors. By placing the tissue anchors around the outside of the annulus and then advancing a smaller-sized heart valve down the array of sutures, the annulus can be constricted simultaneous with delivery of the heart valve, without requiring any further manipulation.

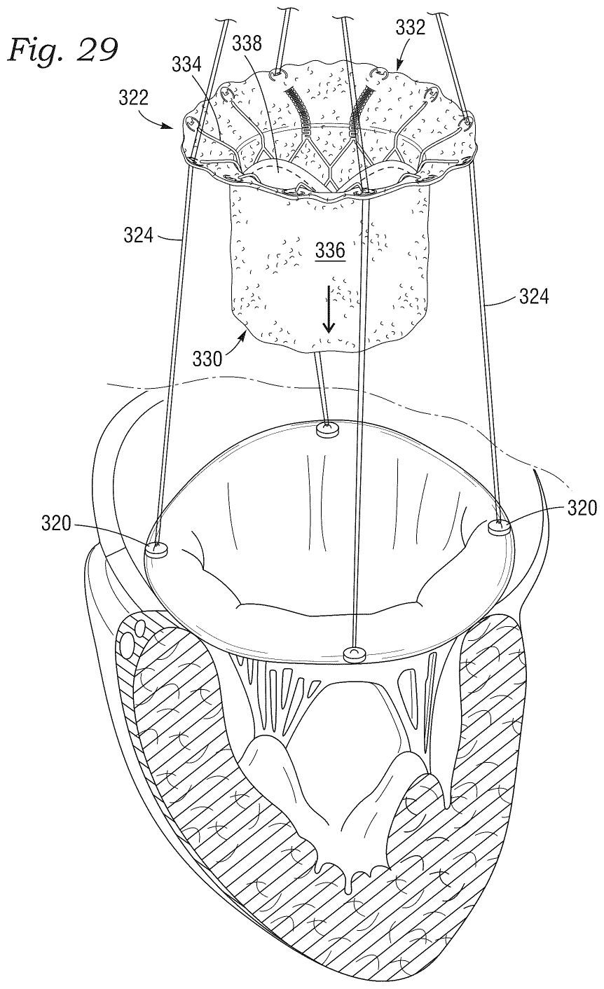

For example, FIG. 29 is a partial sectional view through the left side of a heart showing a mitral valve and annulus with pre-installed tissue anchors 320 therein. An expanded prosthetic heart valve 322 is shown being advanced down an array of guide sutures 324 to the annulus. The heart valve 322 is shown having a configuration much like several described above, with an inner, generally tubular valve portion 330 and a peripheral flange 332 extending radially outward from a proximal end thereof. As before, the valve portion 330 and peripheral flange 332 are desirably formed by a single expandable super-elastic stent 334 covered with fabric 336. Flexible valve leaflets 338 are supported within the valve portion 330 to enable one-way blood flow through the valve 322 (downward for this mitral valve).

Although not shown, the heart valve 322 will be implanted on a beating heart, and as such is first delivered in a collapsed configuration to a position adjacent the target annulus via an access tube or sheath. For example, the heart valve 322 is delivered collapsed through the tube to the left atrium above the mitral annulus, and then expelled from the tube to expanded to its larger shape as seen in FIG. 29. In the same manner, a tricuspid heart valve 322 will be delivered to the right atrium above the tricuspid annulus and expanded. The guide suture 324 pass through the peripheral flange 332 of the valve 322 and are desirably pre-installed and delivered with the valve, with free ends remaining outside the body for manipulation.

The tissue anchors 320 can take a variety of forms, including those having a corkscrew-type lower anchor portion attached to an upper head. Each anchor 320 features a small ring or shackle 340 thereon through which the guide sutures 324 loop. That is, each of the tissue anchors 320 is remotely implanted, with a loop of guide suture 324 pre-attached or subsequently passed through the shackle 340. The tissue anchors 320 are spaced around the annulus so as to define a circumference which is larger than the outer circumference of the peripheral flange 332 of the heart valve 322. As the heart valve 322 is advanced down the array of sutures 324, the disparity in diameter between the peripheral flange 332 and the circumference described by the tissue anchors 320 causes inward constriction of the annulus, as seen in FIG. 30. In FIG. 29, only four coupled tissue anchors 320 guide sutures 324 are shown evenly spaced around the annulus at 90° apart. However, a more uniform constriction of the annulus may be attained using eight or more anchors 320 and sutures 324, most preferably 12 at spaced circumferentially 30° apart.

Each pair of lengths of guide suture 324 passes through the peripheral flange 332 before looping through the shackle 340 of one of the tissue anchors 320. The guide sutures 324 may pass through a suture clip 342 attached to the peripheral flange 332, as will be described in more detail below. Alternatively, the guide sutures 324 may pass through a reinforced region of the peripheral flange 332, and subsequently be used to secure the peripheral flange to the respective tissue anchor 320 by tying a knot, or the like. For example, the heart valve 322 may be advanced down until the peripheral flange 332 is in contact with the target annulus, with the tissue anchors 320 pulled inward and underneath the flange. Each of the pairs of lengths of guide sutures 324 can then be secured on the top side of the peripheral flange 332 using a knot pusher, as is known. However, to facilitate the implantation process, suture clips 342 are preferably incorporated into the peripheral flange 332.

FIG. 30 shows the prosthetic heart valve 322 advanced nearly down to the annulus, causing the pre-installed tissue anchors 320 to be pulled radially inward. At a certain point, the position of the heart valve 322 within the annulus enables the valve leaflets 338 to begin operating and take over the task of regulating blood flow through the annulus from the native leaflets. It is at this point that the surgeon can evaluate the effectiveness of the newly implanted heart valve using fluoroscopy, echocardiography, or the other such visualization techniques. The degree of constriction of the surrounding annulus can vary such that the tissue anchors 320 may remain somewhat separated from the peripheral flange 332 or be cinched completely up against the peripheral flange for maximum constriction, which can also be seen using fluoroscopy and the like. For example, the tissue anchors 320 may be radiopaque with radiopaque elements also sewn into the peripheral flange 332 to compare the relative sizes. As mentioned above, the suture clips 342 may be used to secure the guide sutures 324 at variable stages of annulus constriction. However, as described below, the suture clips 342 may also be used to directly connect to the tissue anchors 320.

The system described with respect to FIGS. 29-30 may operate as extrinsic or intrinsic in terms of the constriction mechanism. That is, if the guide sutures 324 remain connected after the valve starts to function and while the surgeon evaluates the efficacy of the valve implant, further tension or loosening of the sutures 324 is obviously an external actuator and the system is extrinsic. However, if the valve is simply parachuted down into place and the guide sutures 324 severed and tied off, the resulting annulus constriction is done using structures that are carried by or coupled to the heart valve, which is intrinsic to the heart valve.

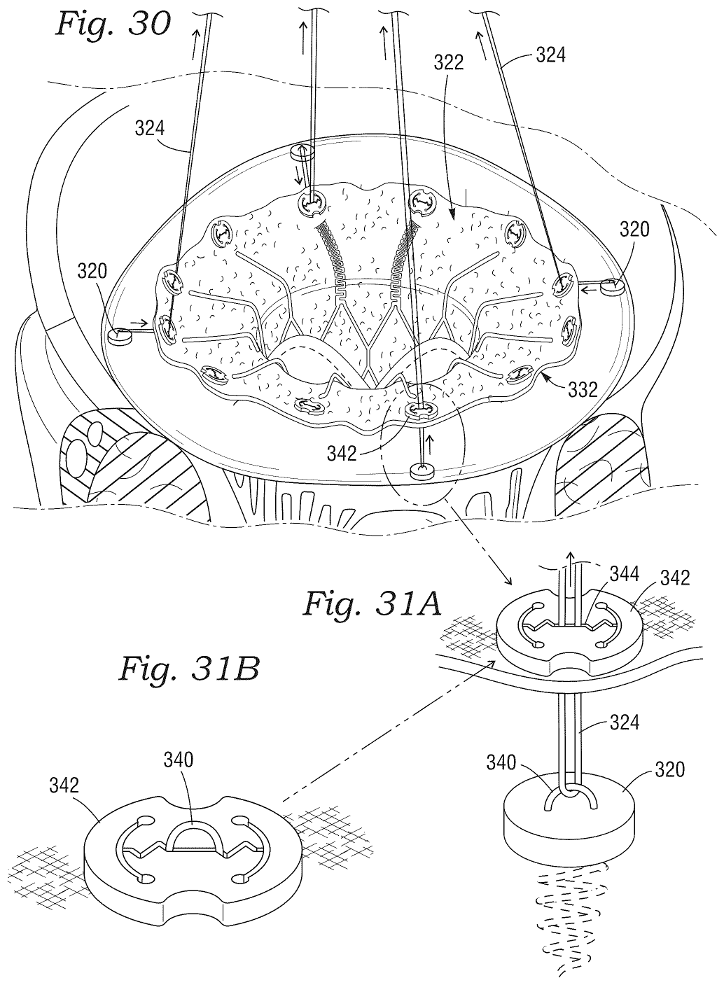

FIGS. 31A and 31B show engagement between the flat suture clip 342 on the peripheral flange 332 of the heart valve 322 and one of the tissue anchors 320. Pulling the guide sutures 324 eventually causes the shackle 340 on the tissue anchor 320 to be pulled upward through an aperture 344 on the suture clip 342. One strand of each guide suture 324 can then be simply pulled free of engagement with the shackle 340 of the anchor 320 and removed from the body.

The clip 342 may be formed of a super-elastic or otherwise resilient metallic material such that the aperture 344 flexes upward to permit one-way passage of the guide sutures 324 and the shackle 340, but resiliently closes to clamp onto either the guide suture 324 or the shackle 340 and prevent reverse movement. The particular suture clip 342 is one as disclosed in U.S. Pat. No. 9,592,048 assigned to Edwards Lifesciences, Inc. of Irvine, CA, the contents of which are hereby expressly incorporated herein. However, a variety of suture clips are known, and accordingly the clips 342 may be differently configured.

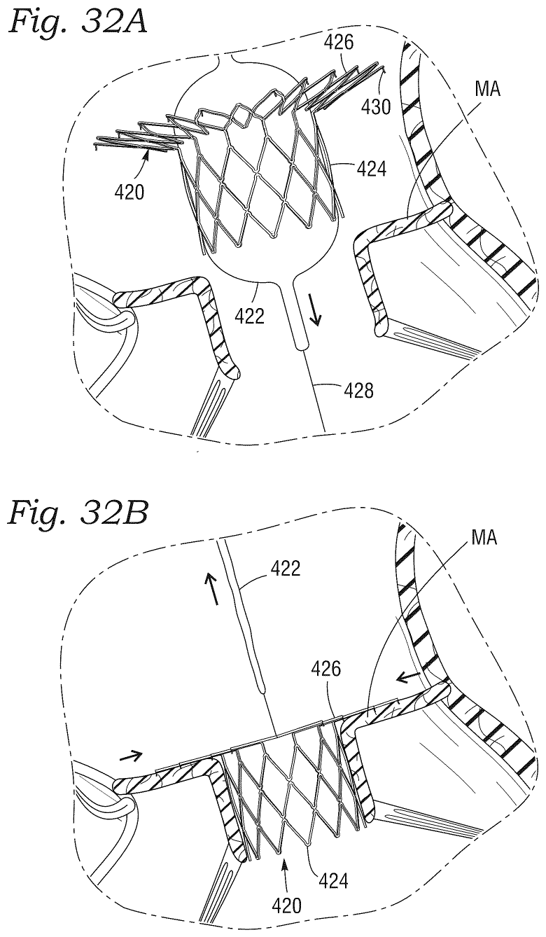

Yet another technique for constricting the annulus post-implant involves the use of a balloon which pre-expands the prosthetic heart valve prior to anchoring at the annulus, followed by removal of the balloon to permit the valve to constrict through its inherent resiliency, thus pulling the annulus inward. For example, FIG. 32A shows a valve stent 420, similar to the stent 100 from FIG. 10A, held in an expanded state by a delivery balloon 422. The overall implant procedure remains via the vasculature with the heart beating, and so expansion of the balloon 422 only occurs once the valve having the valve stent 420 is adjacent the target annulus, in this case the mitral annulus MA. The valve stent 420 has a tubular valve portion 424 and an upper peripheral flange 426. The assembled heart valve may be formed by the single expandable super-elastic stent 420 covered with fabric, and flexible valve leaflets are supported within the valve portion 424 to enable one-way blood flow through the valve (downward for this mitral valve).

The balloon 422 expands the valve portion to about the same diameter as the size of the native valve, which may be distended depending on the particular morphology. The surgeon advances the valve having the valve stent 420 over a guidewire 428 into the annulus such that the tubular valve portion 424 fits closely within the annulus and the peripheral flange 426 engages the annulus on the atrial side. The peripheral flange 426 may have tissue anchors, such as barbs 430 as shown, which pierce the atrial side of the annulus. Alternatively, separate tissue anchors may be deployed around the peripheral flange 426 once it is seated against the annulus.

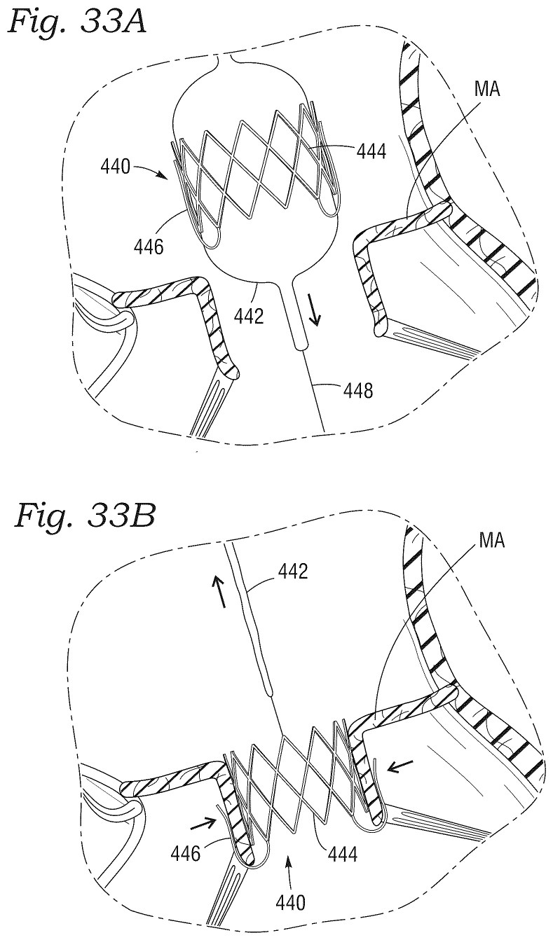

Subsequently, as seen in FIG. 32B, the balloon 422 is deflated and removed from the implant site. This enables the valve stent 420 to assume a smaller relaxed shape, which pulls the annulus inward due to the anchors 430 on the peripheral flange 426. It will also be understood that ventricular anchors such as anchors 106′ seen in FIG. 10D and as described below for FIGS. 33A-33B may also be part of the valve stent 420, to also pull inward the leaflets, though a secondary positioning step is required. Although the balloon 442 is used to first expand and then permit constriction of the valve, the constriction of the annulus is done solely by structures carried by or coupled to the valve, and thus the constriction mechanism is intrinsic to the valve.

FIG. 33A shows another type of heart valve stent 440, similar to the stent 100′ from FIG. 10C, held in an expanded state by a delivery balloon 442 and advancing towards the mitral annulus MA. The overall implant procedure remains via the vasculature with the heart beating, and so expansion of the balloon 442 only occurs once the heart valve having the stent 440 is adjacent to the target annulus.

The valve stent 440 has a tubular valve portion 444 with no upper peripheral flange but ventricular anchors 446 that curl down and back up about 180° from a lower end of the stent main body 444. The ventricular anchors 446 may be configured to constrict post-implant, such as by providing a cinch around the periphery thereof, such as in the embodiment of FIGS. 7-8. The balloon 442 expands the valve portion to about the same diameter as the size of the native valve, which may be distended depending on the particular morphology. The surgeon advances the valve having the valve stent 440 over a guidewire 448 into the annulus such that the tubular valve portion 444 fits closely within the annulus and the ventricular anchors 446 deploy to the outside of the mitral leaflets and are configured to constrict inward, as shown.

Subsequently, as seen in FIG. 33B, the balloon 442 is deflated and removed from the implant site. This enables the valve stent 440 to assume a smaller relaxed shape, which pulls the annulus inward due to the ventricular anchors 446 around the leaflets, as indicated by the movement arrows. Again, this is an intrinsic constricting mechanism.

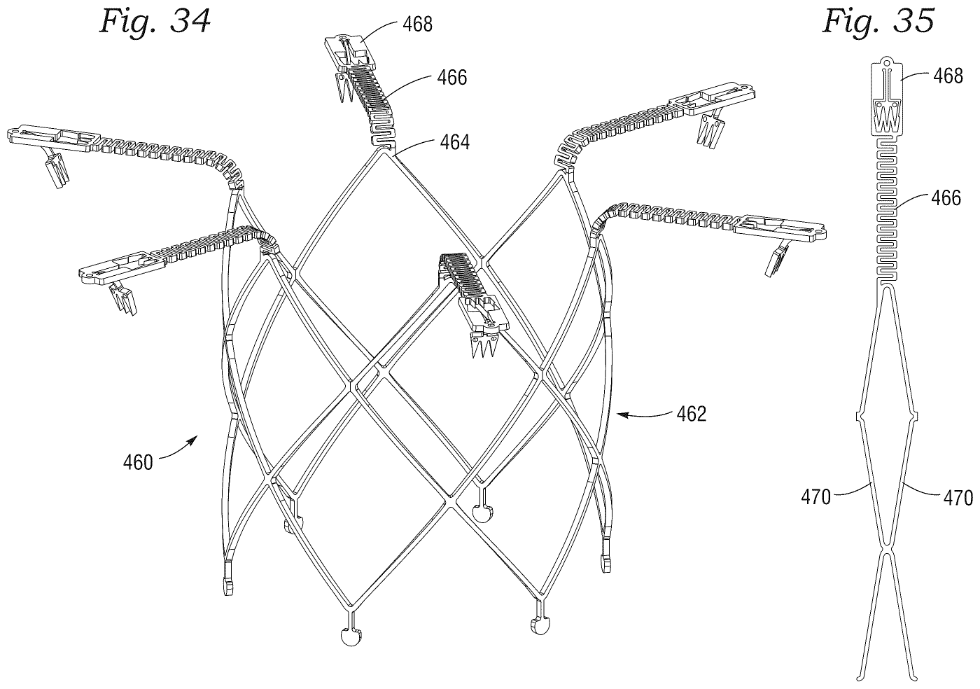

FIG. 34 is a perspective view of a still further heart valve stent 460 having a tubular valve portion 462, illustrated as being formed by struts in a diamond pattern for constriction/expansion. The struts in the valve portion 462 converge at an inflow (upper) end at junctions 464 from which radially extending arms 466 tipped with anchoring barbs 468 extend. The arms 466 radiate outward in a circular pattern to form an upper peripheral flange, and in the illustrated embodiment there are 6 arms, though there may be as few as 4 and more than 6. In the assembled valve the arms 466 are covered in a circular panel of fabric, much like seen at 131 in FIG. 13A.

FIG. 35 is an isolation of one segment of the valve stent 460 with an anchoring arm 466. The struts 470 in the valve portion 462 are shown in an elongated and radially constricted delivery state, and the anchoring arm 466 extends linearly in alignment. As will be described, the super-elastic stent 460 is held in a linear constricted state during transvascular delivery through an access tube or catheter. Once the valve reaches the implant site, the surrounding constraints are removed so that the valve portion 462 expands radially into a tube and the anchoring arms 466 bend radially outward to the generally perpendicular orientation seen in FIG. 34.

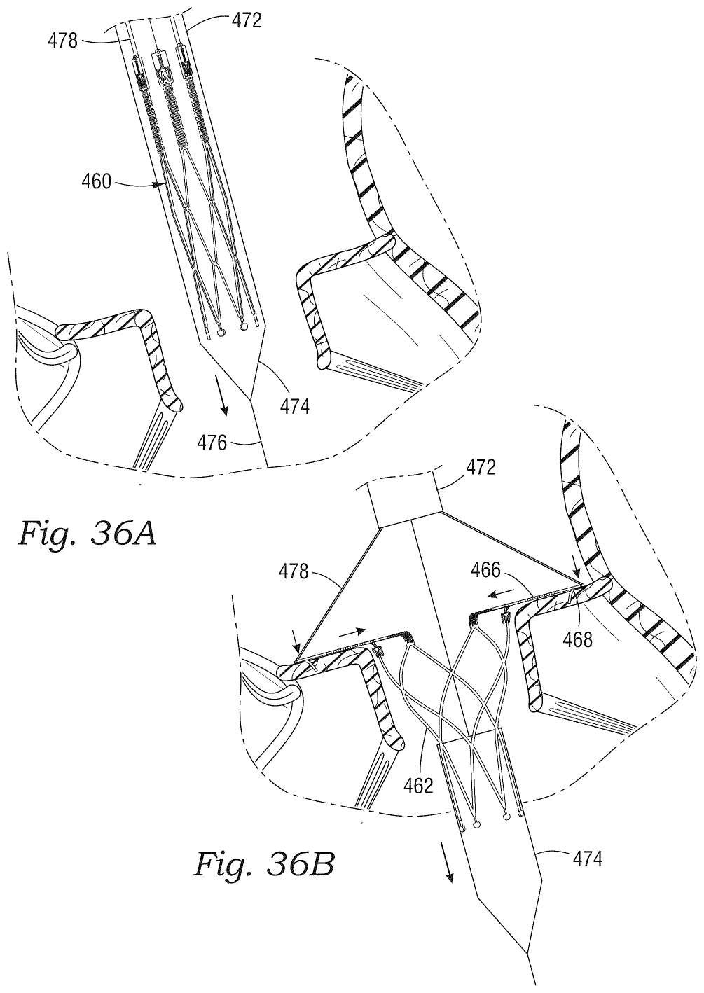

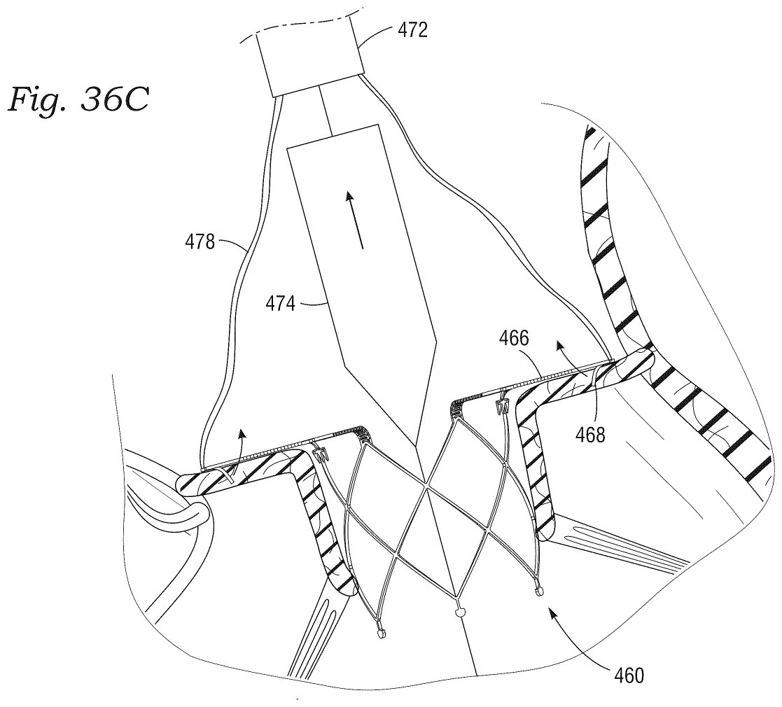

FIGS. 36A-36C show a sequence of delivering and implanting a heart valve having the stent 460 of FIG. 34 through a tubular sheath 472. The sheath 472 may have a tapered nose cone 474 which as will be seen is dis-engageable from a proximal section of the sheath, and the sheath is typically flexible to follow a guidewire 476 pre-installed at the implant site. Trailing sutures or tethers 478 that pass from outside the body through the sheath 472 may be attached to proximal ends of the stent 460, in this case the anchoring barbs 468.

Initially in FIG. 36A, the heart valve is constricted and retained within the tubular sheath 472, at least partially within the tapered nose cone 474. Advancing the sheath 472 over the guidewire 476 brings the nose cone 474 within the target annulus.

FIG. 36B shows detachment and further advancement of the nose cone 474 from the larger sheath 472 past the target annulus. This releases constraint on the anchoring arms 466 first, permitting them to bend perpendicular to the tubular valve portion 462. As will be shown, the anchoring barbs 468 are configured to embed into the tissue of the surrounding annulus, which may be helped by further distal movement of the nose cone 474 using an inner shaft (not shown) of the delivery system. As seen in FIG. 34, the arms 466 have a serpentine configuration which may be initially stretched linearly but which constrict upon release from the delivery sheath 472. This pulls the surrounding annulus inward as indicated by the radial arrows. Of course, any of the various constriction mechanisms described herein may be utilized for the arms 466, such as for example the embodiments described with respect to FIGS. 13-16, or others described herein.

FIG. 36C shows the stent 460 fully released with the tubular valve portion 462 radially expanded within the native valve leaflets and the proximal flange defined by the radially arms 466 anchored in the surrounding tissue. The radial constriction from the arms 466 pulls the annulus inward against the valve portion 462, thus improving anchoring and reducing paravalvular leakage around the valve. The trailing sutures 478 are shown released or loosened, which triggers better anchoring of the barbs 468, described below.

The use of externally controlled sutures 478 in the active barbs 468 means this configuration s a hybrid constriction mechanism, though the radial constricting forces are intrinsic and generated solely by the arms 466. If the barbs 488 were instead passive and simply embedded in the tissue or were activated automatically once implanted, the entire constriction mechanism would be considered intrinsic.

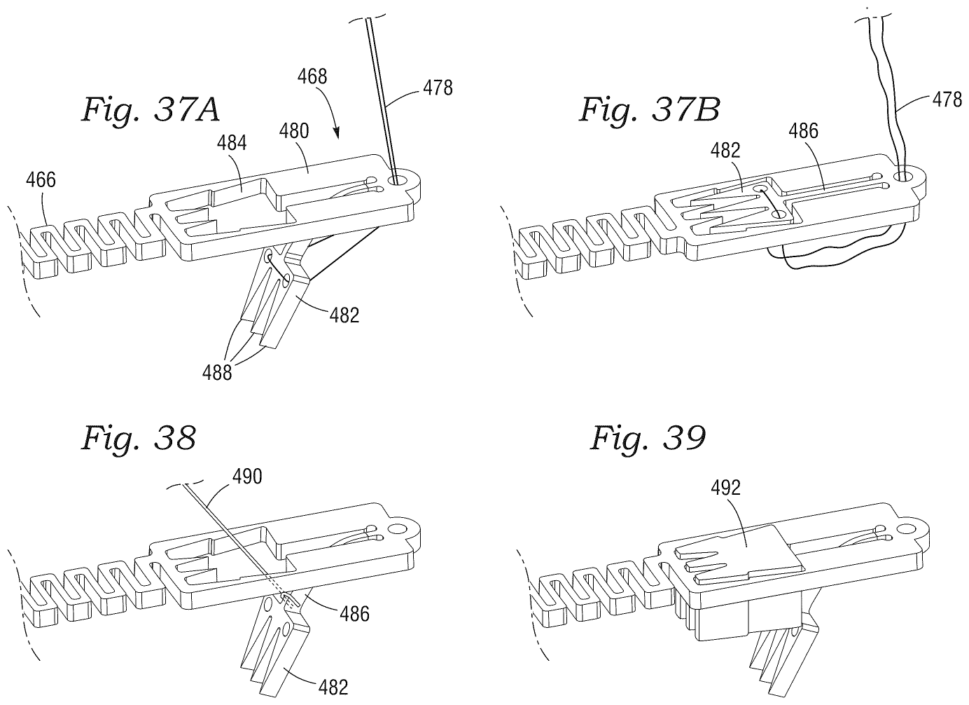

FIGS. 37A and 37B illustrate one configuration for the anchoring barbs 468 of the stent 460 activated by the sutures 478 in tension. Each anchoring barb 468 comprises a flat plate-like member 480 that is oriented to lie against the tissue surrounding the target valve. A flexible portion 482 bends out of the plane of the plate-like member 480 from a similar cut out 484. The flexible portion 482 comprises a thin bending finger 486 cantilevered from within the cut out 484 and terminating in a barbed or forked end 488. In this embodiment, the sutures 478 pass through an outer through hole in the plate-like member 480 and loop though holes formed in the flexible portion 482. Maintaining tension on the sutures 478 bends the flexible portion 482 out of the cut out 484. Once tension in the sutures 478 is released, the flexible portion 482 is permitted to flex back into the cut out 484, as seen in FIG. 37B. The tension may be released by simply allowing one strand of the looped suture 478 to go free, so that the entire suture can then be removed from the stent 460 by pulling on the other strand. As the flexible portion 482 flexes back to its relaxed position, the barbed or forked end 488 embeds deeper into the annulus tissue, thus better anchoring the valve stent 460.

FIGS. 38 and 39 illustrate two alternative configurations for the anchoring barbs of the stent of FIG. 34. In FIG. 38, the tensioning sutures 478 are replaced by a stiff strut 490 having a forked end which engages the flexible portion 482. For example, the forked end may be coupled to the bending finger 486. In this embodiment, the flexible portion 482 is maintained in its bent position by pushing force from the stiff strut 490 but allowed to flex back toward the cut out 484 upon removal of the strut 490. FIG. 39 shows a block of bioresorbable material 492 filling the cut out 484, which forces the flexible portion 482 to remain bent. After a period of implant in the body, such as perhaps 15 minutes, the bioresorbable material 492 dissolves which allows the flexible portion 482 to revert back to its relaxed shape within the cut out 484. The embodiment of FIG. 39 in the stent 460 is an example of a completely intrinsic constriction mechanism.

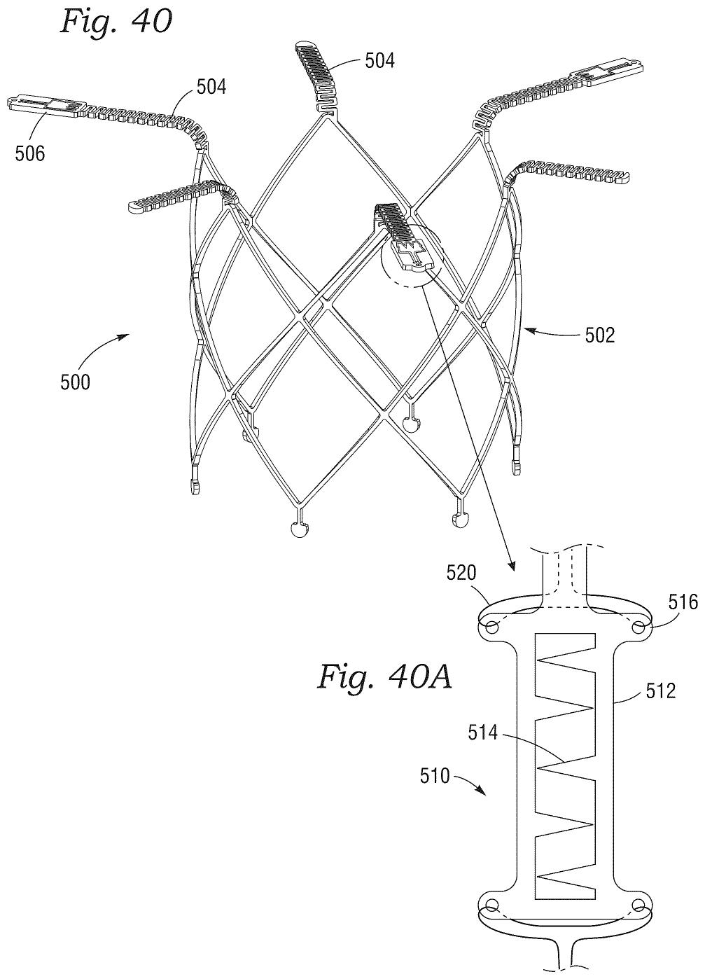

FIG. 40 is a perspective view of a heart valve stent 500 similar to that in FIG. 34 but having anchoring barbs on only some of the radially extending arms. Once again, the stent 500 has a tubular valve portion 502 defining a central axis and within which the valve member with leaflets (not shown) is mounted. A plurality of arms 504 extend radially outward from an inflow end of the valve portion 502 when the stent is expanded. There are 6 arms 504 shown, but only every other arm has an anchoring barb 506 on its outer end. As mentioned above, there may be as few as 4 and more than 6 arms 504, and the anchoring barbs 506 may be distributed differently, such as on every third arm, for example. Preferably, however, the anchoring barbs 506 are distributed evenly circumferentially around the peripheral flange defined by the arms 504.

FIG. 40A is an enlargement of an alternative type of anchoring barb 510 than is shown in FIG. 34 and elsewhere. The anchoring barb 510 has a bear-claw configuration similar to that described above with respect to FIGS. 27-28. The barb 510 comprises a generally rectangular frame member 512 having an opening in a number of inwardly directed teeth 514. Oppositely-projecting levers 516 on both ends of the frame 512 may be bent toward each other using tension sutures 520 to flex the frame 512 and open the jaws defined by the teeth 514. Once tension in the sutures 520 is released, the jaws flex closed again, as was described above. Again, this is a hybrid constriction mechanism where external sutures 520 are used to actuate the barbs 510, but the constricting forces are intrinsic to the valve structure.

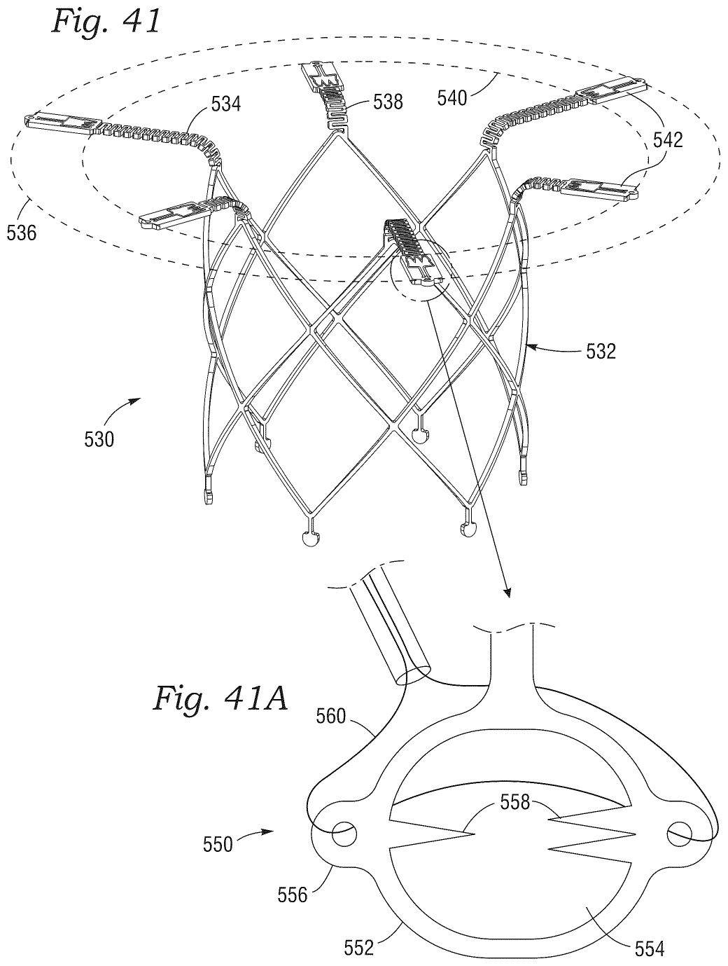

FIG. 41 is a perspective view of still another heart valve stent 530 similar to that in FIG. 34 but having radially extending arms of different lengths. A tubular valve portion 532 connect at upper ends to a plurality of radially extending arms; long arms 534 extending out to a circumference 536, and short arms 538 extending out to a smaller circumference 540. Each of the arms 534, 538 terminates in an anchoring barb 542, though as described above some of the arms may be without anchoring barbs. Once again, the number of arms may vary, and the pattern of short and long arms can also vary from the alternating pattern shown.

FIG. 41A is an enlargement of another type of anchoring barb 550. A generally circular or oval frame 552 defines an opening 554 within which several teeth 558 extend. Oppositely-projecting levers 556 provide through holes for a tension suture 560. By pulling on the suture 560, the levers 526 bend the frame 552 open so that the teeth 558 project in a common direction. Once tension in the suture 560 is released, the teeth 558 come back together again.

The various structures disclosed herein provide enormous potential benefits. For example, shrinking the annulus (rather than pushing outwardly to anchor) would better treat the underlying disease by remodeling the heart to original shape. In addition, pulling the annulus in makes it less likely that the frame will contact the ventricle walls because the final shrunken diameter is smaller. A tighter seal is also created by the constriction, which naturally reduces paravalvular (PV) leakage. In addition, the positive grabbing or anchoring of the inner valve frame to the annulus may eliminate the need for an outer frame, which is used in some current solutions. Finally, shrinking the annulus around the valve means a smaller valve in general than ones that expand outward, which reduces the delivery profile, leading to certain advantages in terms of less traumatic deliveries.

While the foregoing is a complete description of the preferred embodiments of the invention, various alternatives, modifications, and equivalents may be used. Moreover, it will be obvious that certain other modifications may be practiced within the scope of the appended claims.

What is claimed is:

1. A prosthetic heart valve assembly for replacing a native mitral or tricuspid valve, the prosthetic heart valve assembly, comprising:
a self-expandable stent having a main body with an inlet end portion and an outlet end portion, the stent made from a shape memory material and having an annular flange extending radially outwardly from the inlet end portion;
a plurality of anchors configured to engage surrounding tissue and disposed along a surface of the annular flange, wherein the annular flange is adapted to radially constrict the annulus of the native valve upon deployment; and
a valve portion positioned within a passageway of the main body, wherein the valve portion comprises a plurality of leaflets made from pericardium, wherein the valve portion permits flow of blood through the passageway in one direction for replacing the function of the native valve.

2. The prosthetic heart valve assembly of claim 1, wherein the annular flange is coupled to the main body and configured to transition from a large diameter to a small diameter for constricting the annulus of the native valve.

3. The prosthetic heart valve assembly of claim 2, wherein the annular flange comprises a plurality of radial arms, wherein each arm is configured to reduce in length for constricting the annulus of the native valve.

4. The prosthetic heart valve assembly of claim 3, wherein each radial arm includes a bioresorbable material that initially maintains the arm in an elongated state and resorbs in the body for allowing the arm to transition to a shortened state.

5. The prosthetic heart valve assembly of claim 1, wherein the anchors comprise barbs.

6. The prosthetic heart valve assembly of claim 1, wherein the anchors comprise helical screws.

7. The prosthetic heart valve assembly of claim 1, wherein a cinching mechanism connects each of the anchors and wherein the cinching mechanism is adapted to pull the anchors radially inwardly for constricting the annulus of the native valve.

8. The prosthetic heart valve assembly of claim 1, wherein the anchors are not integrated into the heart valve and are deployed separately from the heart valve.

9. The prosthetic heart valve assembly of claim 1, wherein the annular flange comprises a plurality of spirally-arranged arms that when expanded have tips that together define a circle of revolution having a diameter D₁, and wherein rotation of the valve portion about its axis pulls the arms inward such that the tips together define a constricted diameter D₂.

10. The prosthetic heart valve assembly of claim 1, further comprising at least two ventricular anchors extending from the outlet end portion of the main body.

11. The prosthetic heart valve assembly of claim 10, wherein the ventricular anchors are each shaped for capturing a native valve leaflet between the ventricular anchor and the main body of the stent.

12. The prosthetic heart valve assembly of claim 10, wherein the ventricular anchor has an outer surface for engaging surrounding tissue.

13. A prosthetic heart valve assembly for replacing a native mitral or tricuspid valve, the prosthetic heart valve assembly, comprising:
a self-expandable valve stent made from a shape memory material and covered with fabric, the valve stent having a tubular valve portion with an inlet end portion and an outlet end portion and a peripheral flange when expanded formed by an array of struts or arms extending radially outward from and connected to the inlet end portion, the array having an intrinsic radial constriction mechanism and tissue-engaging members; and
a valve portion positioned within a passageway of the main body, wherein the valve portion comprises a plurality of leaflets made from pericardium, wherein the valve portion permits flow of blood through the passageway in one direction for replacing the function of the native valve;
wherein the prosthetic heart valve assembly is adapted to anchor the tissue-engaging members to tissue surrounding the valve annulus to radially constrict and shrink the annulus of the native valve upon deployment.

14. The prosthetic heart valve assembly of claim 13, wherein the array comprises petal-shaped struts coupled to the valve portion such that expansion of the valve portion causes the struts to radially constrict.

15. The prosthetic heart valve assembly of claim 13, wherein the array comprises a plurality of radially-extending arms each of which has a constriction mechanism for reducing a length of the arm built in.

16. The prosthetic heart valve assembly of claim 15, wherein the constriction mechanism includes an extended structure biased to a constricted length and held extended by a bioresorbable suture.

17. The prosthetic heart valve assembly of claim 15, wherein the constriction mechanism includes an extended structure biased to a constricted length and held extended by a stiffening wire.

18. The prosthetic heart valve assembly of claim 15, wherein only some of the radially-extending arms has a tissue-engaging member thereon.

19. The prosthetic heart valve assembly of claim 15, wherein the radially-extending arms have dissimilar lengths.

20. The prosthetic heart valve assembly of claim 13, wherein the array comprises a plurality of spirally-arranged arms that when expanded have tips that together define a circle of revolution having a diameter D₁, and wherein rotation of the valve portion about its axis pulls the arms inward such that the tips together define a constricted diameter D₂.

21. The prosthetic heart valve assembly of claim 13, wherein the valve stent has an outer anchor stent and an inner valve stent, and the array comprises a plurality of radial arms extending from struts connected just at a lower end of the anchor stent, wherein rotation of the anchor stent subsequent to anchoring of barbs at tips of the radial arms causes the struts to rotate into helical shapes and pull the tips inward, the inner valve stent then being expanded within the outer anchor stent.
